SEQUENCE LISTING (142 sequences)

SEQ ID NO: 1 - protein, 142 aa, Artificial Sequence
  Synthetic
MRVQIQFLGLLLLWTSVVQCDVQMTQSPSYLATSPGESVSISCKASKNIDTYLAWYQEKPGKTNKLLIYSGSTLQSGTPSRFSGSGSGTDFTLTIRNLESEDFAVYYCQQHNEYPLTFGSGTKLEIKRADAAPTVSIFPPSS

SEQ ID NO: 2 - protein, 11 aa, Artificial Sequence
  Synthetic
KASKNIDTYLA

SEQ ID NO: 3 - protein, 5 aa, Artificial Sequence
  Synthetic
SGSTL

SEQ ID NO: 4 - protein, 9 aa, Artificial Sequence
  Synthetic
QQHNEYPLT

SEQ ID NO: 5 - protein, 161 aa, Artificial Sequence
  Synthesized
MDTRLCLVFLVLFIKGVQCEVQLVESGGGLVQPGRSLKLSCAASGFTFSNYYMAWVRQAPTKGLEWVASISFEGNRNHYGDSVKGRITISRDNAKSTLYLQMTSLRPEDTATYYCARHRGYSTNFYHDVLDAWGQGALVTVSSAETTAPSVYPLAPGTALK

SEQ ID NO: 6 - protein, 10 aa, Artificial Sequence
  Synthetic
GFTFSNYYMA

SEQ ID NO: 7 - protein, 16 aa, Artificial Sequence
  Synthetic
SISFEGNRNHYGDSVK

SEQ ID NO: 8 - protein, 19 aa, Artificial Sequence
  Synthetic
HRGYSTNFYHDVLDAWGQG

SEQ ID NO: 9 - protein, 111 aa, Artificial Sequence
  Synthesized
DIVLTQSPASLAVSLGQRATISCRASKSVSTSGYSYMYWYQQKPGQPPKLLIYLASNLESGVPARFSGSGSGTDFTLNIHPVEEEDAATYYCQHSRELPFTFGSGTKLEIK

SEQ ID NO: 10 - protein, 15 aa, Artificial Sequence
  Synthetic
RASKSVSTSGYSYMY

SEQ ID NO: 11 - protein, 6 aa, Artificial Sequence
  Synthetic
ASNLES

SEQ ID NO: 12 - protein, 9 aa, Artificial Sequence
  Synthetic
QHSRELPFT

SEQ ID NO: 13 - protein, 121 aa, Artificial Sequence
  Synthesized
QIQLVQSGPELKKPGETVKISCKASGYTFTNYGMNWVKQAPGKGLKWMGWINTYTGEPTYADDFKGRFALSLETSASTAYLQINNLKNEDMATYFCARYRYNKYERAMDYWGQGTSVTVSS

SEQ ID NO: 14 - protein, 10 aa, Artificial Sequence
  Synthetic
GYTFTNYGMN

SEQ ID NO: 15 - protein, 17 aa, Artificial Sequence
  Synthetic
WINTYTGEPTYADDFKG

SEQ ID NO: 16 - protein, 12 aa, Artificial Sequence
  Synthetic
YRYNKYERAMDY

SEQ ID NO: 17 - protein, 112 aa, Artificial Sequence
  Synthesized
DIVMSQSPSSLAVSAGEKVTMSCKSSQSLLNSSTRKNFLAWYQQKPGQSPKLLIYWASTRESGVPDRFTGSGSGTDFTLTISSVQAEDLAVYYCKQSYNRYTFGGGTKLEIK

SEQ ID NO: 18 - protein, 17 aa, Artificial Sequence
  Synthetic
KSSQSLLNSSTRKNFLA

SEQ ID NO: 19 - protein, 7 aa, Artificial Sequence
  Synthetic
WASTRES

SEQ ID NO: 20 - protein, 8 aa, Artificial Sequence
  Synthetic
KQSYNRYT

SEQ ID NO: 21 - protein, 117 aa, Artificial Sequence
  Synthesized
EIQLQQTGPELVKPGASVKISCKASGYSFTDYIMLWVKQSHGKSLEWIGNINPYSGSSGYNLKFKGKATLTVDKSSSTAYMQLNSLTSEDSAVYYCARGKDFAMDYWGQGTSVTVSS

SEQ ID NO: 22 - protein, 10 aa, Artificial Sequence
  Synthetic
GYSFTDYIML

SEQ ID NO: 23 - protein, 17 aa, Artificial Sequence
  Synthetic
NINPYSGSSGYNLKFKG

SEQ ID NO: 24 - protein, 7 aa, Artificial Sequence
  Synthetic
GKDFAMD

SEQ ID NO: 25 - protein, 107 aa, Artificial Sequence
  Synthesized
DIVMTQSPATLSVTPGDRVSLSCRASQSISDYLHWYQQKSHESPRLLIKYASQSISGIPSRFSGSGSGSDFTLSINSVEPEDVGVYYCQNGHNFPRTFGGGTKLEIK

SEQ ID NO: 26 - protein, 11 aa, Artificial Sequence
  Synthetic
RASQSISDYLH

SEQ ID NO: 27 - protein, 7 aa, Artificial Sequence
  Synthetic
YASQSIS

SEQ ID NO: 28 - protein, 9 aa, Artificial Sequence
  Synthetic
QNGHNFPRT

SEQ ID NO: 29 - protein, 122 aa, Artificial Sequence
  Synthesized
QVQLQQPGAELVQPGAPVKLSCKASGYIFTSYWMNWVKQRPGRGLEWIGRIDPSDSKIHYNQKFKDKATLTVDRSSSTAYIQLGSLTSEDSAVYYCAKEGGLRRGDYAMDYWGQGTSVTVSS

SEQ ID NO: 30 - protein, 10 aa, Artificial Sequence
  Synthetic
GYIFTSYWMN

SEQ ID NO: 31 - protein, 17 aa, Artificial Sequence
  Synthetic
RIDPSDSKIHYNQKFKD

SEQ ID NO: 32 - protein, 13 aa, Artificial Sequence
  Synthetic
EGGLRRGDYAMDY

SEQ ID NO: 33 - protein, 112 aa, Artificial Sequence
  Synthesized
DIVMSQSPSSLAVSAGEKVTMNCKSSRSLLNSRIRKNYLAWYQQKPGQSPKLLIYWASTRESGVPDRFTGSGSGTDFTLTISSVQAEDLAVYYCKQSYNLLTFGAGTKLELK

SEQ ID NO: 34 - protein, 17 aa, Artificial Sequence
  Synthetic
KSSRSLLNSRIRKNYLA

SEQ ID NO: 35 - protein, 7 aa, Artificial Sequence
  Synthetic
WASTRES

SEQ ID NO: 36 - protein, 8 aa, Artificial Sequence
  Synthetic
KQSYNLLT

SEQ ID NO: 37 - protein, 120 aa, Artificial Sequence
  Synthesized
DVKLVESGGDLVKPGGSLKLSCAASGFTFSSYIMSWVRQTPEKRLEWVATISSGGSSTYYPDSVKGRFTISRDNAKNTLYLQMSSLKSEDTAMYYCTRDDDYDVKVFAYWGQGTLVTVSA

SEQ ID NO: 38 - protein, 10 aa, Artificial Sequence
  Synthetic
GFTFSSYIMS

SEQ ID NO: 39 - protein, 17 aa, Artificial Sequence
  Synthetic
TISSGGSSTYYPDSVKG

SEQ ID NO: 40 - protein, 11 aa, Artificial Sequence
  Synthetic
DDDYDVKVFAY

SEQ ID NO: 41 - protein, 106 aa, Artificial Sequence
  Synthesized
DIRMTQSPSLLSASVGDRVTLNCKASQNIYNSLAWYQQKLGEGPKVLIFNANSLQTGIPSRFSGSGSGTDFTLTISSLQPEDFATYFCQQFYSGYTFGAGTKLELK

SEQ ID NO: 42 - protein, 106 aa, Artificial Sequence
  Synthetic
DIQVTQSPSLLSASVGDRVTLNCKASQNIYNSLAWYQQKLGEGPKVLIFNANSLQTGIPSRFSGSGSGTDFTLTISSLQPEDFATYFCQQFYSGYTFGAGTKLELK

SEQ ID NO: 43 - protein, 106 aa, Artificial Sequence
  Synthetic
DIVLTQSPSLLSASVGDRVTLNCKASQNIYNSLAWYQQKLGEGPKVLIFNANSLQTGIPSRFSGSGSGTDFTLTISSLQPEDFATYFCQQFYSGYTFGAGTKLELK

SEQ ID NO: 44 - protein, 11 aa, Artificial Sequence
  Synthetic
KASQNIYNSLA

SEQ ID NO: 45 - protein, 7 aa, Artificial Sequence
  Synthetic
NANSLQT

SEQ ID NO: 46 - protein, 8 aa, Artificial Sequence
  Synthetic
QQFYSGYT

SEQ ID NO: 47 - protein, 118 aa, Artificial Sequence
  Synthesized
QVQLKESGPGLVQPSQTLSLTCTVSGFSLTSNGVSWVRQPPGKGLEWIAAISSGGTTYYNSAFKSRLSISRNTSKSQVLLKMNSLQTEDTAMYFCARRYGYGWYFDFWGPGTMVTVSS

SEQ ID NO: 48 - protein, 10 aa, Artificial Sequence
  Synthetic
GFSLTSNGVS

SEQ ID NO: 49 - protein, 16 aa, Artificial Sequence
  Synthetic
AISSGGTTYYNSAFKS

SEQ ID NO: 50 - protein, 10 aa, Artificial Sequence
  Synthetic
RYGYGWYFDF

SEQ ID NO: 51 - protein, 106 aa, Artificial Sequence
  Synthesized
DIRVTQSPSLLSASVGDRVTLNCKGSQNIYKSLAWFRLKRGEAPKLLIYDANSLQTGIPSRFSGSGSGTDFTLTITSLQPEDVATYFCQQYYSGYTFGAGTKLELK

SEQ ID NO: 52 - protein, 106 aa, Artificial Sequence
  Synthesized
DIQVTQSPSLLSASVGDRVTLNCKGSQNIYKSLAWFRLKRGEAPKLLIYDANSLQTGIPSRFSGSGSGTDFTLTITSLQPEDVATYFCQQYYSGYTFGAGTKLELK

SEQ ID NO: 53 - protein, 11 aa, Artificial Sequence
  Synthetic
KGSQNIYKSLA

SEQ ID NO: 54 - protein, 7 aa, Artificial Sequence
  Synthetic
DANSLQT

SEQ ID NO: 55 - protein, 8 aa, Artificial Sequence
  Synthetic
QQYYSGYT

SEQ ID NO: 56 - protein, 116 aa, Artificial Sequence
  Synthesized
QVQLKESGPGLVQSSQTLSLTCTVSGFSLTSNGVSWVRQPPGKGLEWIAAISSGGSTYYNSAFKSRLSISRNTSKSQVLLKMNSLQTEDTGMYFCARHRPFYFDYWGQGVMVTVSS

SEQ ID NO: 57 - protein, 10 aa, Artificial Sequence
  Synthetic
GFSLTSNGVS

SEQ ID NO: 58 - protein, 16 aa, Artificial Sequence
  Synthetic
AISSGGSTYYNSAFKS

SEQ ID NO: 59 - protein, 8 aa, Artificial Sequence
  Synthetic
HRPFYFDY

SEQ ID NO: 60 - protein, 120 aa, Artificial Sequence; identical to SEQ ID NO: 37
  Synthetic

SEQ ID NO: 61 - protein, 120 aa, Artificial Sequence
  Synthetic
EVQLVESGGGLVQPGGSLRLSCAASGFTFSSYIMSWVRQAPGKGLEWVATISSGGSSTYYPDSVKGRFTISRDNAKNSLYLQMNSLRAEDTAVYYCTRDDDYDVKVFAYWGQGTMVTVSS

SEQ ID NO: 62 - protein, 120 aa, Artificial Sequence
  Synthetic
EVQLVESGGGLVQPGGSLRLSCAASGFTFSSYIMSWVRQAPGKRLEWVATISSGGSSTYYPDSVKGRFTISRDNAKNSLYLQMNSLRAEDTAVYYCTRDDDYDVKVFAYWGQGTMVTVSS

SEQ ID NO: 63 - protein, 112 aa, Artificial Sequence; identical to SEQ ID NO: 33
  Synthetic

SEQ ID NO: 64 - protein, 112 aa, Artificial Sequence
  Synthetic
DIVMTQSPDSLAVSLGERATINCKSSRSLLNSRIRKNYLAWYQQKPGQPPKLLIYWASTRESGVPDRFSGSGSGTDFTLTISSLQAEDVAVYYCKQSYNLLTFGQGTKVEIK

SEQ ID NO: 65 - protein, 112 aa, Artificial Sequence
  Synthetic
DIVMTQSPDSLAVSLGERATINCKSSRSLLNSRIRKNYLAWYQQKPGQPPKLLIYWASTRESGVPDRFTGSGSGTDFTLTISSLQAEDVAVYYCKQSYNLLTFGQGTKVEIK

SEQ ID NO: 66 - protein, 116 aa, Artificial Sequence
  Synthetic
QVQLKESGPGLVQSSQTLSLTCTVSGFSLTSNGVSWVRQPPGKGLEWIAAISSGGSTYYNSAFKSRLSISPNTSKSQVLLKMNSLQTEDTGMYFCARHRPFYFDYWGQGVMVTVSS

SEQ ID NO: 67 - protein, 116 aa, Artificial Sequence
  Synthetic
QVTLKESGPVLVKPTETLTLTCTVSGFSLTSNGVSWVRQPPGKALEWIAAISSGGSTYYNSAFKSRLTISRDTSKSQVVLTMTNMDPVDTATYYCARHRPFYFDYWGQGTLVTVSS

SEQ ID NO: 68 - protein, 116 aa, Artificial Sequence
  Synthetic
QVTLKESGPVLVKPTETLTLTCTVSGFSLTSNGVSWVRQPPGKALEWIAAISSGGSTYYNSAFKSRLSISRDTSKSQVVLTMTNMDPVDTATYYCARHRPFYFDYWGQGTLVTVSS

SEQ ID NO: 69 - protein, 116 aa, Artificial Sequence
  Synthetic
QVTLKESGPVLVKPTETLTLTCTVSGFSLTSNGVSWVRQPPGKALEWIAAISSGGSTYYNSAFKSRLTISRNTSKSQVVLTMTNMDPVDTATYYCARHRPFYFDYWGQGTLVTVSS

SEQ ID NO: 70 - protein, 116 aa, Artificial Sequence
  Synthetic
QVTLKESGPVLVKPTETLTLTCTVSGFSLTSSGVSWVRQPPGKALEWIAAISSGGSTYYNSAFKSRLTISPDTSKSQVVLTMTNMDPVDTATYYCARHRPFYFDYWGQGTLVTVSS

SEQ ID NO: 71 - protein, 116 aa, Artificial Sequence
  Synthetic
QVTLKESGPVLVKPTETLTLTCTVSGFSLTSQGVSWVRQPPGKALEWIAAISSGGSTYYNSAFKSRLTISPDTSKSQVVLTMTNMDPVDTATYYCARHRPFYFDYWGQGTLVTVSS

SEQ ID NO: 72 - protein, 116 aa, Artificial Sequence
  Synthetic
QVTLKESGPVLVKPTETLTLTCTVSGFSLTSNAVSWVRQPPGKALEWIAAISSGGSTYYNSAFKSRLTISRDTSKSQVVLTMTNMDPVDTATYYCARHRPFYFDYWGQGTLVTVSS

SEQ ID NO: 73 - protein, 106 aa, Artificial Sequence; identical to SEQ ID NO: 52
  Synthetic

SEQ ID NO: 74 - protein, 106 aa, Artificial Sequence
  Synthetic
DIQMTQSPSSLSASVGDRVTITCKGSQNIYKSLAWFQQKPGKVPKLLIYDANSLQTGIPSRFSGSGSGTDFTLTISSLQPEDVATYYCQQYYSGYTFGGGTKVEIK

SEQ ID NO: 75 - protein, 106 aa, Artificial Sequence
  Synthetic
DIQMTQSPSSLSASVGDRVTITCKGSQNIYKSLAWFQLKPGKVPKLLIYDANSLQTGIPSRFSGSGSGTDFTLTISSLQPEDVATYYCQQYYSGYTFGGGTKVEIK

SEQ ID NO: 76 - protein, 106 aa, Artificial Sequence
  Synthetic
DIQMTQSPSSLSASVGDRVTINCKGSQNIYKSLAWFQQKPGKVPKLLIYDANSLQTGIPSRFSGSGSGTDFTLTISSLQPEDVATYYCQQYYSGYTFGGGTKVEIK

SEQ ID NO: 77 - protein, 118 aa, Artificial Sequence; identical to SEQ ID NO: 47
  Synthetic

SEQ ID NO: 78 - protein, 118 aa, Artificial Sequence
  Synthetic
QVTLKESGPVLVKPTETLTLTCTVSGFSLTSNGVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 79 - protein, 118 aa, Artificial Sequence
  Synthetic
QVTLKESGPVLVKPTETLTLTCTVSGFSLTSNGVSWVRQPPGKALEWIAAISSGGTTYYNSAFKSRLSISRDTSKSQVVLTMTNMDPVDTATYYCARRYGYGWYFDFWGQGTLVTVSS

SEQ ID NO: 80 - protein, 118 aa, Artificial Sequence
  Synthetic
QVTLKESGPVLVKPTETLTLTCTVSGFSLTSSGVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 81 - protein, 118 aa, Artificial Sequence
  Synthetic
QVTLKESGPVLVKPTETLTLTCTVSGFSLTSQGVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 82 - protein, 118 aa, Artificial Sequence
  Synthetic
QVTLKESGPVLVKPTETLTLTCTVSGFSLTSNAVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 83 - protein, 106 aa, Artificial Sequence
  Synthetic
DIRMTQSPSLLSASVGDRVTLNCKASQNIYNSLAWYQQKLGEGPKVLIPNANSLQTGIPSRFSGSGSGTDFTLTISSLQPEDFATYFCQQFYSGYTFGAGTKLELK

SEQ ID NO: 84 - protein, 106 aa, Artificial Sequence
  Synthetic
DIQMTQSPSSLSASVGDRVTITCKASQNIYNSLAWYQQKPGKAPKVLIFNANSLQTGIPSRFSGSGSGTDFTLTISSLQPEDFATYYCQQFYSGYTFGQGTKLEIK

SEQ ID NO: 85 - protein, 106 aa, Artificial Sequence
  Synthetic
DIQMTQSPSSLSASVGDRVTITCKASQNIYNSLAWYQQKPGKAPKVLIFNANSLQTGVPSRFSGSGSGTDFTLTISSLQPEDFATYYCQQFYSGYTFGQGTKLEIK

SEQ ID NO: 86 - protein, 106 aa, Artificial Sequence
  Synthetic
DIQMTQSPSSLSASVGDRVTINCKASQNIYNSLAWYQQKPGKAPKVLIFNANSLQTGIPSRFSGSGSGTDFTLTISSLQPEDFATYYCQQFYSGYTFGQGTKLEIK

SEQ ID NO: 87 - protein, 10 aa, Artificial Sequence
  Synthetic
GFSLTSSGVS

SEQ ID NO: 88 - protein, 10 aa, Artificial Sequence
  Synthetic
GFSLTSQGVS

SEQ ID NO: 89 - protein, 10 aa, Artificial Sequence
  Synthetic
GFSLTSNAVS

SEQ ID NO: 90 - protein, 5 aa, Artificial Sequence
  Synthetic
SNGVS

SEQ ID NO: 91 - protein, 5 aa, Artificial Sequence
  Synthetic
SSGVS

SEQ ID NO: 92 - protein, 10 aa, Artificial Sequence
  Synthetic
RYGYGWYFDF

SEQ ID NO: 93 - protein, 120 aa, Artificial Sequence
  Synthesized
EVKLVESGGGLVQPGGSLRLSCAASGFTFSSYIMSWVRQTPEKRLEWVATISSGGSSTYYPDSVKGRFTISRDNAKNTLYLQMSSLKSEDTAMYYCTRDDDYDVKVFAYWGQGTLVTVSS

SEQ ID NO: 94 - protein, 113 aa, Artificial Sequence
  Synthesized
DIVMTQSPSSLAVSLGERVTINCKSSRSLLNSRIRKNYLAWYQQKPGQSPKLLIYWASTRESGVPDRFSGSGSGTDFTLTISSLQAEDVAVYYCKQSYNLLTFGQGTKVEIKR

SEQ ID NO: 95 - protein, 118 aa, Artificial Sequence
  Synthesized
EVQLKESGPGLVQPSQTLSLTCTVSGFSLTSNGVSWVRQPPGKGLEWIAAISSGGTTYYNSAFKSRLSISRNTSKSQVLLKMNSLQTEDTAMYFCARRYGYGWYFDFWGPGTMVTVSS

SEQ ID NO: 96 - protein, 118 aa, Artificial Sequence
  Synthesized
EVTLKESGPVLVKPTETLTLTCTVSGFSLTSNGVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 97 - protein, 118 aa, Artificial Sequence
  Synthesized
EVTLKESGPVLVKPTETLTLTCTVSGFSLTSNGVSWVRQPPGKALEWIAAISSGGTTYYNSAFKSRLSISRDTSKSQVVLTMTNMDPVDTATYYCARRYGYGWYFDFWGQGTLVTVSS

SEQ ID NO: 98 - protein, 118 aa, Artificial Sequence
  Synthesized
EVTLKESGPVLVKPTETLTLTCTVSGFSLTSSGVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 99 - protein, 118 aa, Artificial Sequence
  Synthesized
EVTLKESGPVLVKPTETLTLTCTVSGFSLTSQGVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 100 - protein, 118 aa, Artificial Sequence
  Synthesized
EVTLKESGPVLVKPTETLTLTCTVSGFSLTSNAVSWVRQPPGKALEWIAAISSGGTTYYNSAFKSRLTISRDTSKSQVVLTMTNMDPVDTATYYCARRYGYGWYFDFWGQGTLVTVSS

SEQ ID NO: 101 - protein, 107 aa, Artificial Sequence [sequence omitted]

SEQ ID NO: 102 - protein, 106 aa, Artificial Sequence
  Synthetic
TVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 103 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNVKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 104 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 105 - protein, 330 aa, Artificial Sequence
  Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 106 - protein, 213 aa, Artificial Sequence
  Synthetic
DIQMTQSPSSLSASVGDRVTINCKASQNIYNSLAWYQQKPGKAPKVLIFNANSLQTGIPSRFSGSGSGTDFTLTISSLQPEDFATYYCQQFYSGYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 107 - protein, 448 aa, Artificial Sequence
  Synthetic
QVTLKESGPVLVKPTETLTLTCTVSGFSLTSNAVSWVRQPPGKALEWIAAISSGGTTYYNSAFKSRLTISRDTSKSQVVLTMTNMDPVDTATYYCARRYGYGWYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 108 - protein, 448 aa, Artificial Sequence
  Synthetic
QVTLKESGPVLVKPTETLTLTCTVSGFSLTSNAVSWVRQPPGKALEWIAAISSGGTTYYNSAFKSRLTISRDTSKSQVVLTMTNMDPVDTATYYCARRYGYGWYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 109 - protein, 448 aa, Artificial Sequence
  Synthetic
EVTLKESGPVLVKPTETLTLTCTVSGFSLTSNAVSWVRQPPGKALEWIAAISSGGTTYYNSAFKSRLTISRDTSKSQVVLTMTNMDPVDTATYYCARRYGYGWYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 110 - protein, 448 aa, Artificial Sequence
  Synthetic
EVTLKESGPVLVKPTETLTLTCTVSGFSLTSNAVSWVRQPPGKALEWIAAISSGGTTYYNSAFKSRLTISRDTSKSQVVLTMTNMDPVDTATYYCARRYGYGWYFDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 219 aa, Artificial Sequence
  Synthetic
DIVMTQSPSSLAVSLGERVTINCKSSRSLLNSRIRKNYLAWYQQKPGQSPKLLIYWASTRESGVPDRFSGSGSGTDFTLTISSLQAEDVAVYYCKQSYNLLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 112 - protein, 450 aa, Artificial Sequence
  Synthetic
EVKLVESGGGLVQPGGSLRLSCAASGFTFSSYIMSWVRQTPEKRLEWVATISSGGSSTYYPDSVKGRFTISRDNAKNTLYLQMSSLKSEDTAMYYCTRDDDYDVKVFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 450 aa, Artificial Sequence
  Synthetic
EVKLVESGGGLVQPGGSLRLSCAASGFTFSSYIMSWVRQTPEKRLEWVATISSGGSSTYYPDSVKGRFTISRDNAKNTLYLQMSSLKSEDTAMYYCTRDDDYDVKVFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 116 aa, Artificial Sequence
  Synthetic
QVQLQQSGAELVKPGASVKISCKASGYAFSTYWMNWVKQAPGEGLEWIGQIYPGDGDTNYNGKFKGRVTLTADKSTSTAYMELSSLRSEDTAVYFCARSDGYYDYWGQGTTVTVSS

SEQ ID NO: 115 - protein, 116 aa, Artificial Sequence
  Synthetic
QVQLQQSGAELVKPGASVKVSCKASGYAFSTYWMNWVRQAPGQGLEWIGQIYPGDGDTNYNGKFKGRVTITADKSTSTAYMELSSLRSEDTAVYFCARSDGYYDYWGQGTTVTVSS

SEQ ID NO: 116 - protein, 116 aa, Artificial Sequence
  Synthetic
EVQLQQSGAELVKPGASVKVSCKASGYAFSTYWMNWVRQAPGQGLEWIGQIYPGDGDTNYNGKFKGRVTITADKSTSTAYMELSSLRSEDTAVYFCARSDGYYDYWGQGTTVTVSS

SEQ ID NO: 117 - protein, 112 aa, Artificial Sequence
  Synthetic
NIVLTQSPASLAVSLGERATISCRASESVDSYGTSFMHWYQQKPGQPPKLLICLASSLESGVPDRFSGSGSRTDFTLTISSLQAEDVATYYCQQNNEDPPTFGQGTKLEIKR

SEQ ID NO: 118 - protein, 112 aa, Artificial Sequence
  Synthetic
NIVLTQSPASLAVSLGERATISCRASESVDSYGTSFMHWYQQKPGQPPKLLISLASSLESGVPDRFSGSGSGTDFTLTISSLQAEDVATYYCQQNNEDPPTFGQGTKLEIKR

SEQ ID NO: 119 - protein, 112 aa, Artificial Sequence
  Synthetic
NIVLTQSPASLAVSLGERATISCRASESVDSYGTSFMHWYQQKPGQPPKLLICLASSLESGVPARFSGSGSRTDFTLTIDPVEAEDAATYYCQQNNEDPPTFGAGTKLEIKR

SEQ ID NO: 120 - protein, 112 aa, Artificial Sequence
  Synthetic
NIVLTQSPASLAVSLGERATISCRASESVDSYGTSFMHWYQQKPGQPPKLLISLASSLESGVPARFSGSGSGTDFTLTISPVEAEDAATYYCQQNNEDPPTFGAGTKLEIKR

SEQ ID NO: 121 - protein, 112 aa, Artificial Sequence
  Synthetic
DIVLTQSPASLAVSLGERATISCRASESVDSYGTSFMHWYQQKPGQPPKLLISLASSLESGVPARFSGSGSGTDFTLTISPLQAEDVATYYCQQNNEDPPTFGQGTKLEIKR

SEQ ID NO: 122 - protein, 112 aa, Artificial Sequence
  Synthetic
DIVLTQSPASLAVSLGERATISCRASESVDSYGTSFMHWYQQKPGQPPKLLISLASSLESGVPARFSGSGSGTDFTLTISPVEAEDAATYYCQQNNEDPPTFGAGTKLEIKR

SEQ ID NO: 123 - protein, 446 aa, Artificial Sequence
  Synthetic
QVQLQQSGAELVKPGASVKISCKASGYAFSTYWMNWVKQAPGEGLEWIGQIYPGDGDTNYNGKFKGRVTLTADKSTSTAYMELSSLRSEDTAVYFCARSDGYYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 124 - protein, 446 aa, Artificial Sequence
  Synthetic
QVQLQQSGAELVKPGASVKISCKASGYAFSTYWMNWVKQAPGEGLEWIGQIYPGDGDTNYNGKFKGRVTLTADKSTSTAYMELSSLRSEDTAVYFCARSDGYYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 125 - protein, 446 aa, Artificial Sequence
  Synthetic
QVQLQQSGAELVKPGASVKVSCKASGYAFSTYWMNWVRQAPGQGLEWIGQIYPGDGDTNYNGKFKGRVTITADKSTSTAYMELSSLRSEDTAVYFCARSDGYYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 126 - protein, 446 aa, Artificial Sequence
  Synthetic
QVQLQQSGAELVKPGASVKVSCKASGYAFSTYWMNWVRQAPGQGLEWIGQIYPGDGDTNYNGKFKGRVTITADKSTSTAYMELSSLRSEDTAVYFCARSDGYYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 127 - protein, 446 aa, Artificial Sequence
  Synthetic
EVQLQQSGAELVKPGASVKVSCKASGYAFSTYWMNWVRQAPGQGLEWIGQIYPGDGDTNYNGKFKGRVTITADKSTSTAYMELSSLRSEDTAVYFCARSDGYYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 128 - protein, 446 aa, Artificial Sequence
  Synthetic
EVQLQQSGAELVKPGASVKVSCKASGYAFSTYWMNWVRQAPGQGLEWIGQIYPGDGDTNYNGKFKGRVTITADKSTSTAYMELSSLRSEDTAVYFCARSDGYYDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPEAAGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 129 - protein, 219 aa, Artificial Sequence
  Synthetic
NIVLTQSPASLAVSLGERATISCRASESVDSYGTSFMHWYQQKPGQPPKLLICLASSLESGVPDRFSGSGSRTDFTLTISSLQAEDVATYYCQQNNEDPPTFGQGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 130 - protein, 218 aa, Artificial Sequence
  Synthetic
NIVLTQSPASLAVSLGERATISCRASESVDSYGTSFMHWYQQKPGQPPKLLICLASSLESGVPDRFSGSGSRTDFTLTISSLQAEDVATYYCQQNNEDPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 131 - protein, 219 aa, Artificial Sequence
  Synthetic
NIVLTQSPASLAVSLGERATISCRASESVDSYGTSFMHWYQQKPGQPPKLLISLASSLESGVPDRFSGSGSGTDFTLTISSLQAEDVATYYCQQNNEDPPTFGQGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 132 - protein, 218 aa, Artificial Sequence
  Synthetic
NIVLTQSPASLAVSLGERATISCRASESVDSYGTSFMHWYQQKPGQPPKLLISLASSLESGVPDRFSGSGSGTDFTLTISSLQAEDVATYYCQQNNEDPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 133 - protein, 219 aa, Artificial Sequence
  Synthetic
NIVLTQSPASLAVSLGERATISCRASESVDSYGTSFMHWYQQKPGQPPKLLICLASSLESGVPARFSGSGSRTDFTLTIDPVEAEDAATYYCQQNNEDPPTFGAGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 134 - protein, 218 aa, Artificial Sequence
  Synthetic
NIVLTQSPASLAVSLGERATISCRASESVDSYGTSFMHWYQQKPGQPPKLLICLASSLESGVPARFSGSGSRTDFTLTIDPVEAEDAATYYCQQNNEDPPTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 135 - protein, 219 aa, Artificial Sequence
  Synthetic
NIVLTQSPASLAVSLGERATISCRASESVDSYGTSFMHWYQQKPGQPPKLLISLASSLESGVPARFSGSGSGTDFTLTISPVEAEDAATYYCQQNNEDPPTFGAGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 136 - protein, 218 aa, Artificial Sequence
  Synthetic
NIVLTQSPASLAVSLGERATISCRASESVDSYGTSFMHWYQQKPGQPPKLLISLASSLESGVPARFSGSGSGTDFTLTISPVEAEDAATYYCQQNNEDPPTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 137 - protein, 219 aa, Artificial Sequence
  Synthetic
DIVLTQSPASLAVSLGERATISCRASESVDSYGTSFMHWYQQKPGQPPKLLISLASSLESGVPARFSGSGSGTDFTLTISPLQAEDVATYYCQQNNEDPPTFGQGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 138 - protein, 218 aa, Artificial Sequence
  Synthetic
DIVLTQSPASLAVSLGERATISCRASESVDSYGTSFMHWYQQKPGQPPKLLISLASSLESGVPARFSGSGSGTDFTLTISPLQAEDVATYYCQQNNEDPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 139 - protein, 219 aa, Artificial Sequence
  Synthetic
DIVLTQSPASLAVSLGERATISCRASESVDSYGTSFMHWYQQKPGQPPKLLISLASSLESGVPARFSGSGSGTDFTLTISPVEAEDAATYYCQQNNEDPPTFGAGTKLEIKRRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 140 - protein, 218 aa, Artificial Sequence
  Synthetic
DIVLTQSPASLAVSLGERATISCRASESVDSYGTSFMHWYQQKPGQPPKLLISLASSLESGVPARFSGSGSGTDFTLTISPVEAEDAATYYCQQNNEDPPTFGAGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 141 - protein, 116 aa, Artificial Sequence
  Synthetic
QVTLKESGPVLVKPTETLTLTCTVSGFSLTSSGVSWVRQPPGKALEWIAAISSGGSTYYNSAFKSRLTISRDTSKSQVVLTMTNMDPVDTATYYCARHRPFYFDYWGQGTLVTVSS

SEQ ID NO: 142 - protein, 116 aa, Artificial Sequence
  Synthetic
QVTLKESGPVLVKPTETLTLTCTVSGFSLTSQGVSWVRQPPGKALEWIAAISSGGSTYYNSAFKSRLTISRDTSKSQVVLTMTNMDPVDTATYYCARHRPFYFDYWGQGTLVTVSS

(19) United States
(12) Patent Application Publication
(10) Pub. No.: US 2017/0002077 A1
(43) Pub. Date: Jan. 5, 2017

(54) COMBINATION TREATMENT FOR MULTIPLE SCLEROSIS

(71) Applicants: PROTHENA BIOSCIENCES LIMITED, DUBLIN (IE); UNIVERSITY OF MUNSTER, Muenster (DE)

(72) Inventors: Stephen Jed Tam, Belmont, CA (US); Theodore Yednock, Forest Knolls, CA (US); Yue Liu, Foster City, CA (US); Nicholas Schwab, Münster (DE); Heinz Wiendl, Münster (DE); Tilman Schneider-Hohendorf, Münster (DE)

(21) Appl. No.: 15/125,568

(22) PCT Filed: Mar. 12, 2015
(86) PCT No.: PCT/IB2015/051785

§ 371 Date: Sep. 12, 2016

Related U.S. Application Data
(60) Provisional application No. 61/952,835, filed on Mar. 13, 2014.
(60) Provisional application No. 62/023,577, filed on Jul. 11, 2014.

(51) Int. Cl. C07K 16/28; C07K 16/30

(52) U.S. Cl. CPC C07K 16/2839; C07K 16/3092; A61K 2039/507

(57) ABSTRACT

The present application provides evidence that a combined blockade against alpha-4 integrin and MCAM is more effective than against either molecule alone. In consequence, the invention provides methods of combination treatment in which both an antagonist of alpha-4 integrin and an MCAM antagonist are administered to a subject having or at risk of multiple sclerosis or other autoimmune disease.

### CROSS-REFERENCES TO RELATED APPLICATIONS

This application claims priority to U.S. Provisional Application No. 61/952,835, filed Mar. 13, 2014, and U.S. Provisional Application No. 62/023,577, filed Jul. 11, 2014, each of the aforementioned applications is incorporated in its entirety herein for all purposes.

### REFERENCE TO A SEQUENCE LISTING, A TABLE, OR A COMPUTER PROGRAM LISTING

The Sequence Listing written in file 455719SEQLIST.txt, created on Mar. 4, 2015, for “COMBINATION TREATMENT FOR MULTIPLE SCLEROSIS” is 153 kilobytes. The information contained in this file is hereby incorporated by reference.

### BACKGROUND

Multiple Sclerosis (MS) is a disease that involves an immune system attack against the central nervous system (brain, spinal cord, and optic nerves). The disease is thought to be triggered in genetically susceptible individuals by a combination of one or more environmental factors. As part of the immune attack on the central nervous system, myelin (the fatty substance that surrounds and protects the nerve fibers in the central nervous system) is damaged, as well as the nerve fibers themselves. The damaged myelin forms scar tissue (sclerosis), which gives the disease its name. When any part of the myelin sheath or nerve fiber is damaged or destroyed, nerve impulses traveling to and from the brain and spinal cord are distorted or interrupted, producing a variety of symptoms.

Although there is no cure for multiple sclerosis, there are now about ten FDA-approved drugs for reducing symptoms, frequency of outbreaks and/or disease progression. These drugs include Aubagio (teriflunomide), Avonex (interferon beta-1a), Betaseron (interferon beta-1b), Copaxone (glatiramer acetate), Extavia (interferon beta-1b), Gilenya (fingolimod), Novantrone (mitoxantrone), Rebif (interferon beta-1a), Tecfidera (dimethyl fumarate), and Tysabri (natalizumab). Natalizumab is one of the more effective of the available drugs. Although natalizumab significantly reduces inflammation and its effects, such as relapses and lesion burden, its effect on progression is less pronounced (Polman et al., N. Engl. J. Med 0.354:899-910 (2006)). Even though the vast majority of subjects reacted favorably to natalizumab treatment, progressive multifocal leukoencephalopathy (PML) has emerged in rare cases (Bloomgren et al., N. Engl. J. Med. 366(20):1870-1880 (2012); Schwab et al., Neurology 78(7):458-467 (2012); Schwab et al., Mult. Scler. 18(3):335-344 (2012)).

### SUMMARY OF THE CLAIMED INVENTION

The invention provides methods of treating or effecting prophylaxis of an autoimmune disease, comprising administering an MCAM antagonist to a subject having or at risk of multiple sclerosis who also receives an alpha-4 integrin antagonist, wherein the MCAM antagonist and alpha-4 integrin antagonist are provided in a regime effective for treatment or prophylaxis of the autoimmune disease. The invention also provides methods of treating or effecting prophylaxis of an autoimmune disease, comprising administering an alpha-4 integrin antagonist to a subject having or at risk of multiple sclerosis who also receives an MCAM-antagonist, wherein the alpha-4 integrin antagonist and the MCAM antagonist are provided in a regime effective for treatment or prophylaxis of the autoimmune disease.

In some methods, the autoimmune disease is multiple sclerosis. In some methods, the alpha-4 integrin antagonist antagonizes alpha-4 integrin binding to VCAM-1. In some methods, the alpha-4 integrin antagonist specifically binds to alpha-4 integrin. In some methods, the alpha-4 integrin antagonist is a monoclonal antibody. In some methods, the monoclonal antibody is natalizumab. In some methods, the MCAM-antagonist antagonizes MCAM binding to laminin-alpha-4. In some methods, the MCAM antagonist is a monoclonal antibody that specifically binds to MCAM. In some methods, the MCAM antagonist is a monoclonal antibody that specifically binds to laminin-alpha-4. In some methods, the MCAM antibody is 1749 or 2120, or a chimeric, veneered or humanized version thereof. In some methods, the alpha-4 integrin antagonist is natalizumab and the MCAM antagonist is 1749 or 2120, or a chimeric, veneered or humanized version thereof. In some methods, the alpha-4 integrin antagonist and MCAM antagonist are provided concurrently such that both at detectable in serum of the subject at the same time. In some methods, the alpha-4 integrin antagonist and MCAM antagonist are provided by simultaneous infusion. In some methods, the alpha-4 integrin antagonist and MCAM antagonist are provided sequentially. In some methods, the alpha-4 integrin antagonist is provided first, the subject develops resistance to the alpha-4 integrin antagonist and the MCAM antagonist is then provided. In some methods, the MCAM antagonist is provided first, the subject develops resistance to the MCAM antagonist and the alpha-4 integrin antagonist is then provided. In some methods, a course of treatment with the alpha-4 integrin antagonist is administered first and the subject has or is at risk of relapsing remitting multiple sclerosis on initiating the course of treatment and the subject has progressed to secondary progressive multiple sclerosis on initiating a course of treatment with the MCAM antagonist. In some methods, the alpha-4 integrin and MCAM antagonists are each provided at intervals of weekly to quarterly. In some methods, the alpha-4 integrin and MCAM antagonists are each provided at four-weekly intervals. In some methods, the dose of each antibody is 50-500 mg/subject. In some methods, the dose of each antibody is 100-200 mg/subject. In some methods, the dose of each antibody is 50-150 mg/subject.

In some methods, the MCAM antibody comprises a mature heavy chain variable region having the amino acid sequence of SEQ ID NO:93 or 100, a mature light chain variable region having the amino acid sequence of SEQ ID NO:86 or 94, a heavy chain constant region having the amino acid sequence of SEQ ID NO:104 or 105, and/or a light chain constant region having the amino acid sequence of SEQ ID NO:101 or 102.

The invention further provides methods of treating or effecting prophylaxis of an autoimmune disease involving T-cell infiltration, comprising administering to a subject having or at risk of the inflammatory disease an alpha-4 antagonist and an MCAM antagonist, wherein the alpha-4 integrin antagonist and the MCAM antagonist are provided in a regime effective for treatment or prophylaxis of the autoimmune disease. In some methods, the autoimmune disease is multiple sclerosis, rheumatoid arthritis, Crohn's disease, inflammatory bowel disease, sarcoidosis, or psoriatic arthritis. In some methods, the MCAM antagonist and alpha-4 integrin antagonist are administered sequentially with the alpha-4 integrin antagonist administered first. In some methods, the MCAM antagonist and alpha-4 antagonist are administered concurrently.

In some methods, the MCAM antagonist is a monoclonal antibody comprising a mature heavy chain variable region having the amino acid sequence of SEQ ID NO:93 or 100, a mature light chain variable region having the amino acid sequence of SEQ ID NO:86 or 94, a heavy chain constant region having the amino acid sequence of SEQ ID NO:104 or 105, and/or a light chain constant region having the amino acid sequence of SEQ ID NO:101 or 102.

### BRIEF DESCRIPTION OF THE DRAWINGS

FIG. 1 (A-C): Changes in the cerebrospinal fluid under long-term treatment with natalizumab reflect a normalization of the central immune response in MS subjects.

FIG. 2 (A-C): CD49d expression in peripheral and central T-cell compartments under long-term natalizumab therapy.

FIGS. 3 (A & B): Natalizumab treatment induces upregulation of PSGL-1.

FIG. 4 (A-K): Molecular distribution of CD49d and PSGL-1 on CD4+ T cells.

FIGS. 5 (A & B): Expression of VCAM-1 and P-selectin on possible routes of entry in multiple sclerosis subject and control tissues.

FIG. 6 (A-G): Influence of natalizumab treatment on rolling and adherence of CD4+ T cells to the ligands of CD49d and PSGL-1 (i.e., VCAM-1/P-selectin).

FIG. 7 (A-G): TH17 cells can use MCAM for firm adhesion to endothelium.

FIG. 8 (A-I): MCAM+ lymphocytes in active MS lesions.

FIG. 9: Schematic overview of the blood-CSF migration of central- and effector memory T cells in MS or under long-term natalizumab therapy.

### BRIEF DESCRIPTION OF THE SEQUENCES

SEQ ID NO:1 is the amino acid sequence of the mature light chain variable region of antibody clone 17.

SEQ ID NO:2 is the amino acid sequence of CDRL1 of the antibody clone 17.

SEQ ID NO:3 is the amino acid sequence of CDRL2 of the antibody clone 17.

SEQ ID NO:4 is the amino acid sequence of CDRL3 of the antibody clone 17.

SEQ ID NO:5 is the amino acid sequence of the mature heavy chain variable region of antibody clone 17.

SEQ ID NO:6 is the amino acid sequence of CDRH1 of the antibody clone 17.

SEQ ID NO:7 is the amino acid sequence of CDRH2 of the antibody clone 17.

SEQ ID NO:8 is the amino acid sequence of CDRH3 of the antibody clone 17.

SEQ ID NO:9 is the amino acid sequence of the mature light chain variable region of antibody 1174.1.3.

SEQ ID NO:10 is the amino acid sequence of CDRL1 of antibody 1174.1.3.

SEQ ID NO:11 is the amino acid sequence of CDRL2 of antibody 1174.1.3.

SEQ ID NO:12 is the amino acid sequence of CDRL3 of antibody 1174.1.3.

SEQ ID NO:13 is the amino acid sequence of the mature heavy chain variable region of antibody 1174.1.3.

SEQ ID NO:14 is the amino acid sequence of CDRH1 of antibody 1174.1.3.

SEQ ID NO:15 is the amino acid sequence of CDRH2 of antibody 1174.1.3.

SEQ ID NO:16 is the amino acid sequence of CDRH3 of antibody 1174.1.3.

SEQ ID NO:17 is the amino acid sequence of the mature light chain variable region of antibody 1414.1.2.

SEQ ID NO:18 is the amino acid sequence of CDRL1 of antibody 1414.1.2.

SEQ ID NO:19 is the amino acid sequence of CDRL2 of antibody 1414.1.2.

SEQ ID NO:20 is the amino acid sequence of CDRL3 of antibody 1414.1.2.

SEQ ID NO:21 is the amino acid sequence of the mature heavy chain variable region of antibody 1414.1.2.

SEQ ID NO:22 is the amino acid sequence of CDRH1 of antibody 1414.1.2.

SEQ ID NO:23 is the amino acid sequence of CDRH2 of antibody 1414.1.2.

SEQ ID NO:24 is the amino acid sequence of CDRH3 of antibody 1414.1.2.

SEQ ID NO:25 is the amino acid sequence of the mature light chain variable region of antibody 1415.1.1.

SEQ ID NO:26 is the amino acid sequence of CDRL1 of antibody 1415.1.1.

SEQ ID NO:27 is the amino acid sequence of CDRL2 of antibody 1415.1.1.

SEQ ID NO:28 is the amino acid sequence of CDRL3 of antibody 1415.1.1.

SEQ ID NO:29 is the amino acid sequence of the mature heavy chain variable region of antibody 1415.1.1.

SEQ ID NO:30 is the amino acid sequence of CDRH1 of antibody 1415.1.1.

SEQ ID NO:31 is the amino acid sequence of CDRH2 of antibody 1415.1.1.

SEQ ID NO:32 is the amino acid sequence of CDRH3 of antibody 1415.1.1.

SEQ ID NO:33 is the amino acid sequence of the mature light chain variable region of antibody 1749.1.3.

SEQ ID NO:34 is the amino acid sequence of CDRL1 of antibody 1749.1.3.

SEQ ID NO:35 is the amino acid sequence of CDRL2 of antibody 1749.1.3.

SEQ ID NO:36 is the amino acid sequence of CDRL3 of antibody 1749.1.3.

SEQ ID NO:37 is the amino acid sequence of the mature heavy chain variable region of antibody 1749.1.3.

SEQ ID NO:38 is the amino acid sequence of CDRH1 of antibody 1749.1.3.

SEQ ID NO:39 is the amino acid sequence of CDRH2 of antibody 1749.1.3.

SEQ ID NO:40 is the amino acid sequence of CDRH3 of antibody 1749.1.3.

SEQ ID NO:41 is the amino acid sequence of the mature light chain variable region of antibody 2120.4.19 version 1.

SEQ ID NO:42 is the amino acid sequence of a mature light chain variable region of antibody 2120.4.19 version 2.

SEQ ID NO:43 is the amino acid sequence of a mature light chain variable region of antibody 2120.4.19 version 3.

SEQ ID NO:44 is the amino acid sequence of CDRL1 of antibody 2120.4.19.

SEQ ID NO:45 is the amino acid sequence of CDRL2 of antibody 2120.4.19.

SEQ ID NO:46 is the amino acid sequence of CDRL3 of antibody 2120.4.19.

SEQ ID NO:47 is the amino acid sequence of the mature heavy chain variable region of antibody 2120.4.19.

SEQ ID NO:48 is the amino acid sequence of CDRH1 of antibody 2120.4.19.

SEQ ID NO:49 is the amino acid sequence of CDRH2 of antibody 2120.4.19.

SEQ ID NO:50 is the amino acid sequence of CDRH3 of antibody 2120.4.19.

SEQ ID NO:51 is the amino acid sequence of the mature light chain variable region of antibody 2107.4.10 version 1.

SEQ ID NO:52 is the amino acid sequence of the mature light chain variable region of antibody 2107.4.10 version 2.

SEQ ID NO:53 is the amino acid sequence of CDRL1 of antibody 2107.4.10.

SEQ ID NO:54 is the amino acid sequence of CDRL2 of antibody 2107.4.10.

SEQ ID NO:55 is the amino acid sequence of CDRL3 of antibody 2107.4.10.

SEQ ID NO:56 is the amino acid sequence of the mature heavy chain variable region of antibody 2107.4.10.

SEQ ID NO:57 is the amino acid sequence of CDRH1 of antibody 2107.4.10.

SEQ ID NO:58 is the amino acid sequence of CDRH2 of antibody 2107.4.10.

SEQ ID NO:59 is the amino acid sequence of CDRH3 of antibody 2107.4.10.

SEQ ID NO:60 is the amino acid sequence of the mature heavy chain variable region of antibody 1749.1.3.

SEQ ID NO:61 is the amino acid sequence of the mature heavy chain variable region of humanized antibody 1749 version 1 (VH1).

SEQ ID NO:62 is the amino acid sequence of the mature heavy chain variable region of humanized antibody 1749 version 2 (VH2).

SEQ ID NO:63 is the amino acid sequence of the mature light chain variable region of antibody 1749.1.3.

SEQ ID NO:64 is the amino acid sequence of the mature light chain variable region of humanized antibody 1749 version 1 (VL1).

SEQ ID NO:65 is the amino acid sequence of the mature light chain variable region of humanized antibody 1749 version 2 (VL2).

SEQ ID NO:66 is the amino acid sequence of the mature heavy chain variable region of antibody 2107.4.10.18.

SEQ ID NO:67 is the amino acid sequence of the mature heavy chain variable region of humanized antibody 2107 version 1 (VH1).

SEQ ID NO:68 is the amino acid sequence of the mature heavy chain variable region of humanized antibody 2107 version 2 (VH2).

SEQ ID NO:69 is the amino acid sequence of the mature heavy chain variable region of humanized antibody 2107 version 3 (VH3).

SEQ ID NO:70 is the amino acid sequence of the mature heavy chain variable region of humanized antibody 2107 version 4A (VH4A).

SEQ ID NO:71 is the amino acid sequence of the mature heavy chain variable region of humanized antibody 2107 version 5A (VH5A).

SEQ ID NO:72 is the amino acid sequence of the mature heavy chain variable region of humanized antibody 2107 version 6 (VH6).

SEQ ID NO:73 is the amino acid sequence of the mature light chain variable region of antibody 2107.4.10.18.

SEQ ID NO:74 is the amino acid sequence of the mature light chain variable region of humanized antibody 2107 version 1 (VL1).

SEQ ID NO:75 is the amino acid sequence of the mature light chain variable region of humanized antibody 2107 version 2 (VL2).

SEQ ID NO:76 is the amino acid sequence of the mature light chain variable region of humanized antibody 2107 version 3 (VL3).

SEQ ID NO:77 is the amino acid sequence of the mature heavy chain variable region of antibody 2120.4.19.6.

SEQ ID NO:78 is the amino acid sequence of the mature heavy chain variable region of humanized antibody 2120 version 1 (VH1).

SEQ ID NO:79 is the amino acid sequence of the mature heavy chain variable region of humanized antibody 2120 version 2 (VH2).

SEQ ID NO:80 is the amino acid sequence of the mature heavy chain variable region of humanized antibody 2120 version 3 (VH3).

SEQ ID NO:81 is the amino acid sequence of the mature heavy chain variable region of humanized antibody 2120 version 4 (VH4).

SEQ ID NO:82 is the amino acid sequence of the mature heavy chain variable region of humanized antibody 2120 version 5 (VH5).

SEQ ID NO:83 is the amino acid sequence of the mature light chain variable region of antibody 2120.4.19.6.

SEQ ID NO:84 is the amino acid sequence of the mature light chain variable region of humanized antibody 2120 version 1 (VL1).

SEQ ID NO:85 is the amino acid sequence of the mature light chain variable region of humanized antibody 2120 version 2 (VL2).

SEQ ID NO:86 is the amino acid sequence of the mature light chain variable region of humanized antibody 2120 version 3 (VL3).

SEQ ID NO:87 is the amino acid sequence of CDRH1 of humanized antibody 2120 version 3 (VH3).

SEQ ID NO:88 is the amino acid sequence of CDRH1 of humanized antibody 2120 version 4 (VH4).

SEQ ID NO:89 is the amino acid sequence of CDRH1 of humanized antibody 2120 version 5 (VH5).

SEQ ID NO:90 is the amino acid sequence of CDRH1 of humanized antibody 2107 version 1 (VH1).

SEQ ID NO:91 is the amino acid sequence of CDRH1 of humanized antibody 2107 version 4 (VH4).

SEQ ID NO:92 is the amino acid sequence of CDRH3 of humanized antibody 2120 version 1-5 (VH1-VH5).

SEQ ID NO:93 is the amino acid sequence of the mature heavy chain variable region of humanized antibody 1749 version 3 (VH3).

SEQ ID NO:94 is the amino acid sequence of the mature light chain variable region of humanized antibody 1749 version 3 (VL3).

SEQ ID NO:95 is the amino acid sequence of the mature heavy chain variable region of antibody 2120.4.19.Q1E, wherein position 1 (Kabat numbering) is occupied by E.

SEQ ID NO:96 is the amino acid sequence of the mature heavy chain variable region of humanized antibody 2120 version 1 Q1E (VH1.Q1E), wherein position 1 (Kabat numbering) is occupied by E.

SEQ ID NO:97 is the amino acid sequence of the mature heavy chain variable region of humanized antibody 2120 version 2 Q1E (VH2.Q1E), wherein position 1 (Kabat numbering) is occupied by E.

SEQ ID NO:98 is the amino acid sequence of the mature heavy chain variable region of humanized antibody 2120 version 3 Q1E (VH3.Q1E), wherein position 1 (Kabat numbering) is occupied by E.

SEQ ID NO:99 is the amino acid sequence of the mature heavy chain variable region of humanized antibody 2120 version 4 Q1E (VH4.Q1E), wherein position 1 (Kabat numbering) is occupied by E.

SEQ ID NO:100 is the amino acid sequence of the mature heavy chain variable region of humanized antibody 2120 version 5 Q1E (VH5.Q1E), wherein position 1 (Kabat numbering) is occupied by E.

SEQ ID NO:101 is the amino acid sequence of a humanized 1749 or 2120 light chain constant region, with Arginine at the N-terminus.

SEQ ID NO:102 is the amino acid sequence of a humanized 1749 or 2120 light chain constant region, without Arginine at the N-terminus.

SEQ ID NO:103 is the amino acid sequence of a humanized 1749 or 2120 heavy chain constant region.

SEQ ID NO:104 is the amino acid sequence of a BIP version heavy chain G1m3 allotype constant region.

SEQ ID NO:105 is the amino acid sequence of a BIP version heavy chain G1m3 allotype constant region.

SEQ ID NO:106 is the amino acid sequence of a mature light chain region of humanized antibody 2120 version 3 (VL3+light chain constant region).

SEQ ID NO:107 is the amino acid sequence of a mature heavy chain region of humanized antibody 2120 version 5 (VH5+BIP version heavy chain G1m3 allotype constant region).

SEQ ID NO:108 is the amino acid sequence of a mature heavy chain region of humanized antibody 2120 version 5 (VH5+BIP version heavy chain G1m3 allotype constant region).

SEQ ID NO:109 is the amino acid sequence of a mature heavy chain region of humanized antibody 2120 version 5 Q1E (VH5.Q1E+BIP version heavy chain G1m3 allotype constant region).

SEQ ID NO:110 is the amino acid sequence of a mature heavy chain region of humanized antibody 2120 version 5 Q1E (VH5.Q1E+BIP version heavy chain G1m3 allotype constant region).

SEQ ID NO:111 is the amino acid sequence of a mature light chain region of humanized antibody 1749 version 3 (VL3+light chain constant region).

SEQ ID NO:112 is the amino acid sequence of a mature heavy chain region of humanized antibody 1749 version 3 (VH3+BIP version heavy chain G1m3 allotype constant region).

SEQ ID NO:113 is the amino acid sequence of a mature heavy chain region of humanized antibody 1749 version 3 (VH3+BIP version heavy chain G1m3 allotype constant region).

SEQ ID NO:114 is the amino acid sequence of humanized 19C12 heavy chain variable region version 1 (H1).

SEQ ID NO:115 is the amino acid sequence of humanized 19C12 heavy chain variable region version 2 (H2).

SEQ ID NO:116 is the amino acid sequence of humanized 19C12 heavy chain variable region version 3 (H3).

SEQ ID NO:117 is the amino acid sequence of humanized 19C12 light chain variable region version 1 (L1).

SEQ ID NO:118 is the amino acid sequence of humanized 19C12 light chain variable region version 2 (L2).

SEQ ID NO:119 is the amino acid sequence of humanized 19C12 light chain variable region version 3 (L3).

SEQ ID NO:120 is the amino acid sequence of humanized 19C12 light chain variable region version 4 (L4).

SEQ ID NO:121 is the amino acid sequence of humanized 19C12 light chain variable region version 5 (L5).

SEQ ID NO:122 is the amino acid sequence of humanized 19C12 light chain variable region version 6 (L6).

SEQ ID NO:123 is the amino acid sequence of exemplary mature heavy chain of 19C12 humanized antibody (H1+BIP version heavy chain G1m3 allotype constant region).

SEQ ID NO:124 is the amino acid sequence of exemplary mature heavy chain of 19C12 humanized antibody (H1+BIP version heavy chain G1m3 allotype constant region).

SEQ ID NO:125 is the amino acid sequence of exemplary mature heavy chain of 19C12 humanized antibody (H2+BIP version heavy chain G1m3 allotype constant region).

SEQ ID NO:126 is the amino acid sequence of exemplary mature heavy chain of 19C12 humanized antibody (H2+BIP version heavy chain G1m3 allotype constant region).

SEQ ID NO:127 is the amino acid sequence of exemplary mature heavy chain of 19C12 humanized antibody (H3+BIP version heavy chain G1m3 allotype constant region).

SEQ ID NO:128 is the amino acid sequence of exemplary mature heavy chain of 19C12 humanized antibody (H3+BIP version heavy chain G1m3 allotype constant region).

SEQ ID NO:129 is the amino acid sequence of exemplary mature light chain of 19C12 humanized antibody (L1+light chain constant region with arginine at N-terminus).

SEQ ID NO:130 is the amino acid sequence of exemplary mature light chain of 19C12 humanized antibody (L1+light chain constant region without arginine at N-terminus).

SEQ ID NO:131 is the amino acid sequence of exemplary mature light chain of 19C12 humanized antibody (L2+light chain constant region with arginine at N-terminus).

SEQ ID NO:132 is the amino acid sequence of exemplary mature light chain of 19C12 humanized antibody (L2+light chain constant region without arginine at N-terminus).

SEQ ID NO:133 is the amino acid sequence of exemplary mature light chain of 19C12 humanized antibody (L3+light chain constant region with arginine at N-terminus).

SEQ ID NO:134 is the amino acid sequence of exemplary mature light chain of 19C12 humanized antibody (L3+light chain constant region without arginine at N-terminus).

SEQ ID NO:135 is the amino acid sequence of exemplary mature light chain of 19C12 humanized antibody (L4+light chain constant region with arginine at N-terminus).

SEQ ID NO:136 is the amino acid sequence of exemplary mature light chain of 19C12 humanized antibody (L4+light chain constant region without arginine at N-terminus).

SEQ ID NO:137 is the amino acid sequence of exemplary mature light chain of 19C12 humanized antibody (L5+light chain constant region with arginine at N-terminus).

SEQ ID NO:138 is the amino acid sequence of exemplary mature light chain of 19C12 humanized antibody (L5+light chain constant region without arginine at N-terminus).

SEQ ID NO:139 is the amino acid sequence of exemplary mature light chain of 19C12 humanized antibody (L6+light chain constant region with arginine at N-terminus).

SEQ ID NO:140 is the amino acid sequence of exemplary mature light chain of 19C12 humanized antibody (L6+light chain constant region without arginine at N-terminus).

SEQ ID NO:141 is the amino acid sequence of the mature heavy chain variable region of humanized antibody 2107 version 4B (VH4B).

SEQ ID NO:142 is the amino acid sequence of the mature heavy chain variable region of humanized antibody 2107 version 5B (VH5B).

### DEFINITIONS

Antagonists for treatment, such as monoclonal antibodies, are typically provided in isolated form. This means that an monoclonal antibody or other antagonist is typically at least 50% w/w pure of proteins and other macromolecules arising from its production or purification but does not exclude the possibility that the monoclonal antibody or other antagonist is combined with an excess of pharmaceutical acceptable carrier(s) or other vehicle intended to facilitate its use. Sometimes monoclonal antibodies or other antagonists are at least 60%, 70%, 80%, 90%, 95 or 99% w/w pure of proteins and other macromolecules from production or purification.

Specific binding of a monoclonal antibody to its target antigen means an affinity of at least 10⁶, 10⁷, 10⁸, 10⁹, or 10¹⁰ M⁻¹. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that a monoclonal antibody binds one and only one target.

The basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. The carboxy-terminal portion of each chain defines a constant region primarily responsible for effector function.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” region of about 12 or more amino acids, with the heavy chain also including a “D” region of about 10 or more amino acids. (See generally, Fundamental Immunology (Paul, W., ed., 2nd ed. Raven Press, N.Y., 1989, Ch. 7, incorporated by reference in its entirety for all purposes).

The mature variable regions of each light/heavy chain pair form the antibody binding site. Thus, an intact antibody has two binding sites. Except in bifunctional or bispecific antibodies, the two binding sites are the same. The chains all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the domains FR1, CDR1, FR2, CDR2, FR3, CDR3 and FR4. The assignment of amino acids to each domain is in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987); Chothia et al., Nature 342:878-883 (1989). Kabat also provides a widely used numbering convention (Kabat numbering) in which corresponding residues between different heavy chains or between different light chains are assigned the same number (e.g., H83 means position 83 by Kabat numbering in the mature heavy chain variable region; likewise position L36 means position 36 by Kabat numbering in the mature light chain variable region). Kabat numbering is used throughout in referring to positions in the variable region of an antibody unless explicitly stated otherwise.

The term “antibody” includes intact antibodies and binding fragments thereof. Typically, fragments compete with the intact antibody from which they were derived for specific binding to the target including separate heavy chains, light chains Fab, Fab′, F(ab′)₂, F(ab)c, diabodies, Dabs, nanobodies, and Fv. Fragments can be produced by recombinant DNA techniques, or by enzymatic or chemical separation of intact immunoglobulins. The term “antibody” also includes a bispecific antibody and/or a humanized antibody. A bispecific or bifunctional antibody is an artificial hybrid antibody having two different heavy/light chain pairs and two different binding sites (see, e.g., Songsivilai and Lachmann, Clin. Exp. Immunol., 79:315-321 (1990); Kostelny et al., J. Immunol. 148:1547-53 (1992).

The term “epitope” refers to a site on an antigen to which an antibody binds. An epitope can be formed from contiguous amino acids or noncontiguous amino acids juxtaposed by tertiary folding of one or more proteins. Epitopes formed from contiguous amino acids are typically retained on exposure to denaturing solvents whereas epitopes formed by tertiary folding are typically lost on treatment with denaturing solvents. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539; Carter, U.S. Pat. No. 6,407,213; Adair, U.S. Pat. Nos. 5,859,205 and 6,881,557; Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having its CDRs, preferably as defined by Kabat, entirely or substantially from a donor antibody and variable region framework sequences and constant regions, if present, entirely or substantially from human antibody sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85, 90, 95 or 100% of corresponding residues defined by Kabat are identical.

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody, and are about two-thirds human sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions.

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2×, 5×, 10×, 20× or 100×) inhibits binding of the reference antibody by at least 50%, for example, 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

A “subject” includes a human or other mammalian subject that receives either prophylactic or therapeutic treatment.

Percentage sequence identities are determined with antibody sequences maximally aligned by the Kabat numbering convention. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises antibody may contain the antibody alone or in combination with other ingredients.

Designation of a range of values includes all integers within or defining the range, and all subranges defined by integers within the range.

Unless otherwise apparent from the context, the term “about” encompasses values within a standard margin of error of measurement (SEM) of a stated value.

An individual is at increased risk of a disease if the subject has at least one known risk-factor (e.g., genetic, biochemical, family history, situational exposure) placing individuals with that risk factor at a statistically significant greater risk of developing the disease than individuals without the risk factor.

The term “symptom” refers to a subjective evidence of a disease, such as altered gait, as perceived by the subject. A “sign” refers to objective evidence of a disease as observed by a physician.

Co-administration of a pharmacological antagonists means that the antagonists are administered sufficiently close in time for detectable amounts of the antagonists to present in the plasma simultaneously and/or the antagonists exert a treatment effect on the same episode of disease or the antagonists act co-operatively, or synergistically in treating the same episode of disease.

Statistically significant refers to a p-value that is <0.05, preferably <0.01 and most preferably <0.001.

A “small molecule” is defined herein to have a molecular weight below about 600, preferably below about 1000 daltons. Generally, a small molecule is a non-peptide small organic molecule.

### DETAILED DESCRIPTION

### I. General

Although natalizumab substantially inhibits extravasation of T-cells, particularly TH1 cells, into the CNS, via its binding to integrin alpha-4 integrin, some T-cells (T_{H}17) still enter the CNS mediated by MCAM interactions with its ligand lamin-alpha-4. The present application provides evidence a combined blockade against alpha-4 integrin and MCAM is more effective against extravasation by these cells than either molecule alone. Based in part on this result, the invention provides methods of combination treatment in which both an antagonist of alpha-4 integrin and an MCAM antagonist are administered to a subject having or at risk of multiple sclerosis. The combination of agents can offer the potential for cooperative or synergistic effects, and in consequence greater efficacy, reduced side effects and/or an improved therapeutic window compared with use of individual antagonists.

II. Target Molecules and their Ligands

Alpha-4 integrin is a component of heterodimeric integrins alpha4 beta1 (VLA-4) and alpha4 beta7. Its ligands include VCAM-1 and fibronectin. Swiss Prot Accession numbers for exemplary forms of the human proteins are as follows: alpha-4, P13612, vcam-1, P19320.

MCAM (melanoma cell adhesion molecule, also known as CD146 and MUC18) refers to a cell surface glycoprotein belonging to the immunoglobulin superfamily involved in cell adhesion, and in cohesion of the endothelial monolayer at intercellular junctions in vascular tissue. It also promotes tumor progression of many cancers including melanoma and prostate cancer. It is known to interact in a homotypic/homophilic manner and may also bind to other ligands. The human MCAM includes five immunoglobulin domains (1: amino acid residues 19-129; 2: amino acid residues 139-242; 3: amino acid residues 244-321; 4: amino acid residues 335-424; and 5: amino acid residues 430-510).

Laminin α4 refers to one of the polypeptide chains found in laminin molecules, which are expressed in the basal lamina (of the basement membrane), a protein network foundation for most cells and organs. Laminins are known to bind to cell membranes through plasma membrane molecules and contribute to cell attachment. The laminin α4 chain typically forms a complex with a laminin β-chain, and a laminin γ-chain. The laminin α4 chain is found in numerous laminin molecules including laminin 411 (laminin 8 or α4β1γ1); laminin 421 (laminin 9 or α4β2γ1), and laminin 423 (laminin 14 or α4β2γ3). There are two main isoforms of the human laminin α4-chain: GenBank Accession Nos. NP001098676 and NP001098677. “Laminin 411” refers to a trimeric polypeptide complex made up of three polypeptide subunits or chains: α4-chain, a β1-chain, and a γ1-chain.

Unless otherwise apparent from the context reference to alpha-4 integrin, VCAM-1, fibronectin, MCAM and laminin-alpha-4 refers toa natural human sequenece of such proteins, usually excluding the siginal peptide if present. Swiss Prot references for exemplary human sequences of MCAM and its ligand Laminin-alpha-4 are P43121 and Q16363, respectively.

### III. Exemplary Antagonists

Antagonist against alpha-4 integrin or MCAM include antibodies, fusion proteins of receptors or ligands to an IgG constant region other biologic binding molecules, and small molecules. Antibodies can be monoclonal or polyclonal. Antibodies can be nonhuman, such as mouse or rat, nonhuman primate or can be human. Antibodies can be chimeric, veneered, humanized, primatized and the like.

Non-antibody binding molecules include, for example, anticalins, which are based upon the lipocalin scaffold, a protein structure characterized by a rigid beta-barrel that supports four hypervariable loops which form the ligand binding site. Novel binding specificities are engineered by targeted random mutagenesis in the loop regions, in combination with functional display and guided selection (Skerra (2008) FEBS J. 275: 2677-2683). Other suitable scaffolds may include, for example, adnectins, or monobodies, based on the tenth extracellular domain of human fibronectin III (Koide and Koide (2007) Methods Mol. Biol. 352: 95-109); affibodies, based on the Z domain of staphylococcal protein A (Nygren et al. (2008) FEBS J. 275: 2668-2676)); DARPins, based on ankyrin repeat proteins (Stumpp et al. (2008) Drug. Discov. Today 13: 695-701); fynomers, based on the SH3 domain of the human Fyn protein kinase (Grabulovski et al. (2007) J. Biol. Chem. 282: 3196-3204); affitins, based on Sac7d from Sulfolobus acidolarius (Krehenbrink et al. (2008) J. Mol. Biol. 383: 1058-1068); affilins, based on human y-B-crystallin (Ebersbach et al. (2007) J. Mol. Biol. 372: 172-185); avimers, based on the A domains of membrane receptor proteins (Silverman et al. (2005) Biotechnol. 23: 1556-1561); cysteine-rich knottin peptides (Kolmar (2008) FEBS J. 275: 2684-2690); and engineered Kunitz-type inhibitors (Nixon and Wood (2006) Curr. Opin. Drug. Discov. Dev. 9: 261-268). For review, see Gebauer and Skerra (2009) Curr. Opin. Chem. Biol. 13: 245-255.

Reference to an alpha-4 antagonist or an alpha-4 integrin antagonist refers to an antagonist, such as an antibody, that binds to alpha-4 integrin and inhibits its interaction as a component of alpha-4 beta-1 or alpha-4-beta-7 with a ligand, such as VCAM-1 and fibronectin, or which binds to VCAM-1 or fibronectin and inhibits the interaction of such molecule with alpha-4 integrin. Some alpha-4 integrin antagonists specifically bind to alpha-4 irrespective whether isolated or a component of a heterodimeric integrin, such as alpha-4 beta 1 or alpha-4 beta-7. Other alpha-4 integrin antagonists specifically bind alpha-4 integrin only as a component of a heterodimeric integrin, such as alpha-4 beta-1 or alpha-4 beta-7.

A preferred alpha-4 integrin antagonist is natalizumab, an IgG4 humanized antibody that specifically binds alpha-4 integrin, and inhibits binding of alpha-4 beta 1 and alpha-4 beta 7 to VCAM-1 and/or fibronectin. The antibody is FDA-approved and commercially available. Several mouse anti-VLA-4 monoclonal antibodies such as HP1/2 and other anti-VLA-4 antibodies (e.g., mAb HP2/1, HP2/4, L25, P4C2, P4G9) have been described previously (see, for example, Pulido et al. (1991) J. BIOL. CHEM. 266(16):10241-5 and Fryer et al. (1997) J. CLIN. INVEST. 99: 2036-2044). Humanized, chimeric or veneered versions of such antibodies can be used. Antibodies competing for binding to alpha-4 integrin with natalizumab can also be used, as can other humanized antibodies incorporating the same Kabat CDRs as natalizumab. Antagonists also include VCAM-1 fusion proteins, VCAM-1/Ig fusion proteins, anti-VLA-4 antibodies, and fibronectin peptides preferably containing the amino acid sequence EILDV. Several small molecules have been already proposed as VLA-4 and/or LPAM-1 antagonists. They are described WO96/22966, WO98/53817, WO01/14328, WO99/06431, WO99/06432, WO99/06436, WO99/10312, WO99/48879, WO00/18759, WO00/20396, WO99/36393, WO99/52898, WO99/62901, WO00/67746 and WO02/08206 and U.S. Pat. No. 7,361,679. Those compounds have a urea structure or phenylalanine structure, or 2-phenyl-3-heteroarylpropionic acid structure.

Antagonist activity can be assayed by several assays to determine the concentration of an antagonist required to block the binding of VLA-4- or VLA-7 expressing cells (for example, Ramos cells, Jurkat cells, A375 melanoma cells, as well as human peripheral blood lymphocytes (PBL)) to fibronectin or VCAM-1 coated plates. For example, once the wells of a microtiter plate are coated with either fibronectin or soluble VCAM-1, varying concentrations of the test antagonist then are added together with appropriately labeled VLA-4-expressing cells. Alternatively, the test antagonist may be added first and allowed to incubate with the coated wells prior to the addition of the labeled VLA-4-expressing cells. The cells are incubated in the wells for at least 30 minutes. Following incubation, the wells are emptied and washed Inhibition of binding is measured by quantitating the fluorescence or radioactivity bound to the plate for each of the various concentrations of test compound, as well as for controls containing no test compound.

An MCAM antagonist refers to an antagonist that fully or partially inhibits the ability of MCAM (i) to specifically bind its ligand: a laminin α4 chain, e.g., the α4 chain of laminin 411; and/or (ii) to facilitate an MCAM-expressing cell, e.g., a T_{H}17 cell, to infiltrate into or migrate to a subject's tissue. MCAM antagonists include antibodies or other antagonists binding to MCAM or to its ligand laminin alpha-4.

Preferred antagonists against MCAM are antibodies described in WO 2012/170071 and PCT/US2013/058773 filed Sep. 9, 2013, particularly the antibodies designated clone 17 in WO 2012/170071 and the mouse anti-human MCAM monoclonal clones designated 1174.1.3, 1414.1.2, 1415.1.1, and 1749.1.3, and the rat anti-human MCAM monoclonal antibody clones designated 2120.4.19 and 2107.4.10 described in PCT/US2013/058773. Variants of monoclonal antibody clone 2120.4.19 are described in U.S. Application No. 61/952,116 filed Mar. 12, 2014. Chimeric, veneered or humanized forms of these antibodies comprising the Kabat CDRs of anyone of the antibodies, or otherwise disclosed in the WO 2012/170071, International Application No. PCT/US2013/058773, in U.S. Application No. 61/952,123 filed Mar. 12, 2014 or U.S. Application No. 61/952,116 filed Mar. 12, 2014 can also be used. Antibodies competing with any of these antibodies for binding to human MCAM can also be used. Preferred antibodies bind to an epitope on MCAM including residue 141 or residue 318.

A preferred anti-MCAM antibody is designated clone 17 comprising a mature light chain variable region having the amino acid sequence designated SEQ ID NO:1 and a mature heavy chain variable region having the amino acid sequence designated SEQ ID NO:5.

Amino acid sequence of the mature light chain variable region of antibody clone 17.

(SEQ ID NO: 1)

MRVQIQFLGLLLLWTSVVQCDVQMTQSPSYLATSPGESVSISCKASKNID
TYLAWYQEKPGKTNKLLIYSGSTLQSGTPSRFSGSGSGTDFTLTIRNLES
EDFAVYYCQQHNEYPLTFGSGTKLEIKRADAAPTVSIFPPSS

Amino acid sequence of CDRL1 of the antibody clone 17.

(SEQ ID NO: 2)

KASKNIDTYLA

Amino acid sequence of CDRL2 of the antibody clone 17.

(SEQ ID NO: 3)

SGSTL

Amino acid sequence of CDRL3 of the antibody clone 17.

(SEQ ID NO: 4)

QQHNEYPLT

Amino acid sequence of the mature heavy chain variable region of antibody clone 17.

(SEQ ID NO: 5)

MDTRLCLVFLVLFIKGVQCEVQLVESGGGLVQPGRSLKLSCAASGFTFSN
YYMAWVRQAPTKGLEWVASISFEGNRNHYGDSVKGRITISRDNAKSTLYL
QMTSLRPEDTATYYCARHRGYSTNFYHDVLDAWGQGALVTVSSAETTAPS
VYPLAPGTALK

Amino acid sequence of CDRH1 of the antibody clone 17.

(SEQ ID NO: 6)

GFTFSNYYMA

Amino acid sequence of CDRH2 of the antibody clone 17.

(SEQ ID NO: 7)

SISFEGNRNHYGDSVK

Amino acid sequence of CDRH3 of the antibody clone 17.

(SEQ ID NO: 8)

HRGYSTNFYHDVLDAWGQG

A preferred anti-MCAM antibody is mouse anti-human MCAM monoclonal clones designated 1174.1.3 comprising a mature light chain variable region having the amino acid sequence designated SEQ ID NO:9 and a mature heavy chain variable region having the amino acid sequence designated SEQ ID NO:13.

Amino acid sequence of the mature light chain variable region of antibody 1174.1.3.

(SEQ ID NO: 9)

DIVLTQSPASLAVSLGQRATISCRASKSVSTSGYSYMYWYQQKPGQPPKL
LIYLASNLESGVPARFSGSGSGTDFTLNIHPVEEEDAATYYCQHSRELPF
TFGSGTKLEIK

Amino acid sequence of CDRL1 of antibody 1174.1.3.

(SEQ ID NO: 10)

RASKSVSTSGYSYMY

Amino acid sequence of CDRL2 of antibody 1174.1.3.

(SEQ ID NO: 11)

ASNLES

Amino acid sequence of CDRL3 of antibody 1174.1.3.

(SEQ ID NO: 12)

QHSRELPFT

Amino acid sequence of the mature heavy chain variable region of antibody 1174.1.3.

(SEQ ID NO: 13)

QIQLVQSGPELKKPGETVKISCKASGYTFTNYGMNWVKQAPGKGLKWMGW
INTYTGEPTYADDFKGRFALSLETSASTAYLQINNLKNEDMATYFCARYR
YNKYERAMDYWGQGTSVTVSS

Amino acid sequence of CDRH1 of antibody 1174.1.3.

(SEQ ID NO: 14)

GYTFTNYGMN

Amino acid sequence of CDRH2 of antibody 1174.1.3.

(SEQ ID NO: 15)

WINTYTGEPTYADDFKG

Amino acid sequence of CDRH3 of antibody 1174.1.3.

(SEQ ID NO: 16)

YRYNKYERAMDY

A preferred anti-MCAM antibody is mouse anti-human MCAM monoclonal clone designated 1414.1.2 comprising a mature light chain variable region having the amino acid sequence designated SEQ ID NO:17 and a mature heavy chain variable region having the amino acid sequence designated SEQ ID NO:21.

Amino acid sequence of the mature light chain variable region of antibody 1414.1.2.

(SEQ ID NO: 17)

DIVMSQSPSSLAVSAGEKVTMSCKSSQSLLNSSTRKNFLAWYQQKPGQSP
KLLIYWASTRESGVPDRFTGSGSGTDFTLTISSVQAEDLAVYYCKQSYNR
YTFGGGTKLEIK

Amino acid sequence of CDRL1 of antibody 1414.1.2.

(SEQ ID NO: 18)

KSSQSLLNSSTRKNFLA

Amino acid sequence of CDRL2 of antibody 1414.1.2.

(SEQ ID NO: 19)

WASTRES

Amino acid sequence of CDRL3 of antibody 1414.1.2.

(SEQ ID NO: 20)

KQSYNRYT

Amino acid sequence of the mature heavy chain variable region of antibody 1414.1.2.

(SEQ ID NO: 21)

EIQLQQTGPELVKPGASVKISCKASGYSFTDYIMLWVKQSHGKSLEWIGN
INPYSGSSGYNLKFKGKATLTVDKSSSTAYMQLNSLTSEDSAVYYCARGK
DFAMDYWGQGTSVTVSS

Amino acid sequence of CDRH1 of antibody 1414.1.2.

(SEQ ID NO: 22)

GYSFTDYIML

Amino acid sequence of CDRH2 of antibody 1414.1.2.

(SEQ ID NO: 23)

NINPYSGSSGYNLKFKG

Amino acid sequence of CDRH3 of antibody 1414.1.2.

(SEQ ID NO: 24)

GKDFAMD

A preferred anti-MCAM antibody is mouse anti-human MCAM monoclonal clone designated 1415.1.1 comprising a mature light chain variable region having the amino acid sequence designated SEQ ID NO:25 and a mature heavy chain variable region having the amino acid sequence designated SEQ ID NO:29.

Amino acid sequence of the mature light chain variable region of antibody 1415.1.1.

(SEQ ID NO: 25)

DIVMTQSPATLSVTPGDRVSLSCRASQSISDYLHWYQQKSHESPRLLIKY
ASQSISGIPSRFSGSGSGSDFTLSINSVEPEDVGVYYCQNGHNFPRTFGG
GTKLEIK

Amino acid sequence of CDRL1 of antibody 1415.1.1.

(SEQ ID NO: 26)

RASQSISDYLH

Amino acid sequence of CDRL2 of antibody 1415.1.1.

(SEQ ID NO: 27)

YASQSIS

Amino acid sequence of CDRL3 of antibody 1415.1.1.

(SEQ ID NO: 28)

QNGHNFPRT

Amino acid sequence of the mature heavy chain variable region of antibody 1415.1.1.

(SEQ ID NO: 29)

QVQLQQPGAELVQPGAPVKLSCKASGYIFTSYWMNWVKQRPGRGLEWIGR
IDPSDSKIHYNQKFKDKATLTVDRSSSTAYIQLGSLTSEDSAVYYCAKEG
GLRRGDYAMDYWGQGTSVTVSS

Amino acid sequence of CDRH1 of antibody 1415.1.1.

(SEQ ID NO: 30)

GYIFTSYWMN

Amino acid sequence of CDRH2 of antibody 1415.1.1.

(SEQ ID NO: 31)

RIDPSDSKIHYNQKFKD

Amino acid sequence of CDRH3 of antibody 1415.1.1.

(SEQ ID NO: 32)

EGGLRRGDYAMDY

A preferred anti-MCAM antibody is mouse anti-human MCAM monoclonal clone designated 1749.1.3 comprising a mature light chain variable region having the amino acid sequence designated SEQ ID NO:33 and a mature heavy chain variable region having the amino acid sequence designated SEQ ID NO:37.

Amino acid sequence of the mature light chain variable region of antibody 1749.1.3.

(SEQ ID NO: 33)

DIVMSQSPSSLAVSAGEKVTMNCKSSRSLLNSRIRKNYLAWYQQKPGQSP
KLLIYWASTRESGVPDRFTGSGSGTDFTLTISSVQAEDLAVYYCKQSYNL
LTFGAGTKLELK

Amino acid sequence of CDRL1 of antibody 1749.1.3.

(SEQ ID NO: 34)

KSSRSLLNSRIRKNYLA

Amino acid sequence of CDRL2 of antibody 1749.1.3.

(SEQ ID NO: 35)

WASTRES

Amino acid sequence of CDRL3 of antibody 1749.1.3.

(SEQ ID NO: 36)

KQSYNLLT

Amino acid sequence of the mature heavy chain variable region of antibody 1749.1.3.

(SEQ ID NO: 37)

DVKLVESGGDLVKPGGSLKLSCAASGFTFSSYIMSWVRQTPEKRLEWVAT
ISSGGSSTYYPDSVKGRFTISRDNAKNTLYLQMSSLKSEDTAMYYCTRDD
DYDVKVFAYWGQGTLVTVSA

Amino acid sequence of CDRH1 of antibody 1749.1.3.

(SEQ ID NO: 38)

SYIMS

Amino acid sequence of CDRH2 of antibody 1749.1.3.

(SEQ ID NO: 39)

TISSGGSSTYYPDSVKG

Amino acid sequence of CDRH3 of antibody 1749.1.3.

(SEQ ID NO: 40)

DDDYDVKVFAY

A preferred anti-MCAM antibody is rat anti-human MCAM monoclonal antibody designated 2120.4.19.

Amino acid sequence of the mature light chain variable region of antibody 2120.4.19 version 1.

(SEQ ID NO: 41)

DIRMTQSPSLLSASVGDRVTLNCKASQNIYNSLAWYQQKLGEGPKVLIFN
ANSLQTGIPSRFSGSGSGTDFTLTISSLQPEDFATYFCQQFYSGYTFGAG
TKLELK

Amino acid sequence of a mature light chain variable region of antibody 2120.4.19 version 2.

(SEQ ID NO: 42)

DIQVTQSPSLLSASVGDRVTLNCKASQNIYNSLAWYQQKLGEGPKVLIFN
ANSLQTGIPSRFSGSGSGTDFTLTISSLQPEDFATYFCQQFYSGYTFGAG
TKLELK

Amino acid sequence of a mature light chain variable region of antibody 2120.4.19 version 3.

(SEQ ID NO: 43)

DIVLTQSPSLLSASVGDRVTLNCKASQNIYNSLAWYQQKLGEGPKVLIFN
ANSLQTGIPSRFSGSGSGTDFTLTISSLQPEDFATYFCQQFYSGYTFGAG
TKLELK

Amino acid sequence of CDRL1 of antibody 2120.4.19.

(SEQ ID NO: 44)

KASQNIYNSLA

Amino acid sequence of CDRL2 of antibody 2120.4.19.

(SEQ ID NO: 45)

NANSLQT

Amino acid sequence of CDRL3 of antibody 2120.4.19.

(SEQ ID NO: 46)

QQFYSGYT

Amino acid sequence of the mature heavy chain variable region of antibody 2120.4.19.

(SEQ ID NO: 47)

QVQLKESGPGLVQPSQTLSLTCTVSGFSLTSNGVSWVRQPPGKGLEWIAA
ISSGGTTYYNSAFKSRLSISRNTSKSQVLLKMNSLQTEDTAMYFCARRYG
YGWYFDFWGPGTMVTVSS

Amino acid sequence of CDRH1 of antibody 2120.4.19.

(SEQ ID NO: 48)

GFSLTSNGVS

Amino acid sequence of CDRH2 of antibody 2120.4.19.

(SEQ ID NO: 49)

AISSGGTTYYNSAFKS

Amino acid sequence of CDRH3 of antibody 2120.4.19.

(SEQ ID NO: 50)

RYGYGWYFDF

A preferred anti-MCAM antibody is rat anti-human MCAM monoclonal antibody designated 2107.4.10.

Amino acid sequence of the mature light chain variable region of antibody 2107.4.10 version 1.

(SEQ ID NO: 51)

DIRVTQSPSLLSASVGDRVTLNCKGSQNIYKSLAWFRLKRGEAPKLLIY
DANSLQTGIPSRFSGSGSGTDFTLTITSLQPEDVATYFCQQYYSGYTFG
AGTKLELK

Amino acid sequence of the mature light chain variable region of antibody 2107.4.10 version 2.

(SEQ ID NO: 52)

DIQVTQSPSLLSASVGDRVTLNCKGSQNIYKSLAWFRLKRGEAPKLLIY
DANSLQTGIPSRFSGSGSGTDFTLTITSLQPEDVATYFCQQYYSGYTFG
AGTKLELK

Amino acid sequence of CDRL1 of antibody 2107.4.10.

(SEQ ID NO: 53)

KGSQNIYKSLA

Amino acid sequence of CDRL2 of antibody 2107.4.10.

(SEQ ID NO: 54)

DANSLQT

Amino acid sequence of CDRL3 of antibody 2107.4.10.

(SEQ ID NO: 55)

QQYYSGYT

Amino acid sequence of the mature heavy chain variable region of antibody 2107.4.10.

(SEQ ID NO: 56)

QVQLKESGPGLVQSSQTLSLTCTVSGFSLTSNGVSWVRQPPGKGLEWIAA
ISSGGSTYYNSAFKSRLSISRNTSKSQVLLKMNSLQTEDTGMYFCARHRP
FYFDYWGQGVMVTVSS

Amino acid sequence of CDRH1 of antibody 2107.4.10.

(SEQ ID NO: 57)

GFSLTSNGVS

Amino acid sequence of CDRH2 of antibody 2107.4.10.

(SEQ ID NO: 58)

AISSGGSTYYNSAFKS

Amino acid sequence of CDRH3 of antibody 2107.4.10.

(SEQ ID NO: 59)

HRPFYFDY

A preferred version of the humanized 1749 antibody is version VH2/VL2 comprising a mature heavy chain variable region having the amino acid sequence designated SEQ ID NO:62 and a mature light chain variable region having the amino acid sequence designated SEQ ID NO:65.

Amino acid sequence of the mature heavy chain variable region of antibody 1749.1.3.

(SEQ ID NO: 60)

DVKLVESGGDLVKPGGSLKLSCAASGFTFSSYIMSWVRQTPEKRLEWVAT
ISSGGSSTYYPDSVKGRFTISRDNAKNTLYLQMSSLKSEDTAMYYCTRDD
DYDVKVFAYWGQGTLVTVSA

Amino acid sequence of the mature heavy chain variable region of humanized antibody 1749 version 1 (VH1).

(SEQ ID NO: 61)

EVQLVESGGGLVQPGGSLRLSCAASGFTFSSYIMSWVRQAPGKGLEWVAT
ISSGGSSTYYPDSVKGRFTISRDNAKNSLYLQMNSLRAEDTAVYYCTRDD
DYDVKVFAYWGQGTMVTVSS

Amino acid sequence of the mature heavy chain variable region of humanized antibody 1749 version 2 (VH2).

(SEQ ID NO: 62)

EVQLVESGGGLVQPGGSLRLSCAASGFTFSSYIMSWVRQAPGKRLEWVAT
ISSGGSSTYYPDSVKGRFTISRDNAKNSLYLQMNSLRAEDTAVYYCTRDD
DYDVKVFAYWGQGTMVTVSS

Amino acid sequence of the mature light chain variable region of antibody 1749.1.3.

(SEQ ID NO: 63)

DIVMSQSPSSLAVSAGEKVTMNCKSSRSLLNSRIRKNYLAWYQQKPGQSP
KLLIYWASTRESGVPDRFTGSGSGTDFTLTISSVQAEDLAVYYCKQSYNL
LTFGAGTKLELK

Amino acid sequence of the mature light chain variable region of humanized antibody 1749 version 1 (VL1).

(SEQ ID NO: 64)

DIVMTQSPDSLAVSLGERATINCKSSRSLLNSRIRKNYLAWYQQKPGQPP
KLLIYWASTRESGVPDRFSGSGSGTDFTLTISSLQAEDVAVYYCKQSYNL
LTFGQGTKVEIK

Amino acid sequence of the mature light chain variable region of humanized antibody 1749 version 2 (VL2).

(SEQ ID NO: 65)

DIVMTQSPDSLAVSLGERATINCKSSRSLLNSRIRKNYLAWYQQKPGQPP
KLLIYWASTRESGVPDRFTGSGSGTDFTLTISSLQAEDVAVYYCKQSYNL
LTFGQGTKVEIK

Amino acid sequence of the mature heavy chain variable region of antibody 2107.4.10.18.

(SEQ ID NO: 66)

QVQLKESGPGLVQSSQTLSLTCTVSGFSLTSNGVSWVRQPPGKGLEWIAA
ISSGGSTYYNSAFKSRLSISPNTSKSQVLLKMNSLQTEDTGMYFCARHRP
FYFDYWGQGVMVTVSS

Amino acid sequence of the mature heavy chain variable region of humanized antibody 2107 version 1 (VH1).

(SEQ ID NO: 67)

QVTLKESGPVLVKPTETLTLTCTVSGFSLTSNGVSWVRQPPGKALEWIAA
ISSGGSTYYNSAFKSRLTISRDTSKSQVVLTMTNMDPVDTATYYCARHRP
FYFDYWGQGTLVTVSS

Amino acid sequence of the mature heavy chain variable region of humanized antibody 2107 version 2 (VH2).

(SEQ ID NO: 68)

QVTLKESGPVLVKPTETLTLTCTVSGFSLTSNGVSWVRQPPGKALEWIAA
ISSGGSTYYNSAFKSRLSISRDTSKSQVVLTMTNMDPVDTATYYCARHRP
FYFDYWGQGTLVTVSS

Amino acid sequence of the mature heavy chain variable region of humanized antibody 2107 version 3 (VH3).

(SEQ ID NO: 69)

QVTLKESGPVLVKPTETLTLTCTVSGFSLTSNGVSWVRQPPGKALEWIAA
ISSGGSTYYNSAFKSRLTISRNTSKSQVVLTMTNMDPVDTATYYCARHRP
FYFDYWGQGTLVTVSS

Amino acid sequence of the mature heavy chain variable region of humanized antibody 2107 version 4A (VH4A).

(SEQ ID NO: 70)

QVTLKESGPVLVKPTETLTLTCTVSGFSLTSSGVSWVRQPPGKALEWIAA
ISSGGSTYYNSAFKSRLTISPDTSKSQVVLTMTNMDPVDTATYYCARHRP
FYFDYWGQGTLVTVSS

Amino acid sequence of the mature heavy chain variable region of humanized antibody 2107 version 4B (VH4B).

(SEQ ID NO: 141)

QVTLKESGPVLVKPTETLTLTCTVSGFSLTSSGVSWVRQPPGKALEWIAA
ISSGGSTYYNSAFKSRLTISRDTSKSQVVLTMTNMDPVDTATYYCARHRP
FYFDYWGQGTLVTVSS

Amino acid sequence of the mature heavy chain variable region of humanized antibody 2107 version 5A (VH5A).

(SEQ ID NO: 71)

QVTLKESGPVLVKPTETLTLTCTVSGFSLTSQGVSWVRQPPGKALEWIAA
ISSGGSTYYNSAFKSRLTISPDTSKSQVVLTMTNMDPVDTATYYCARHRP
FYFDYWGQGTLVTVSS

Amino acid sequence of the mature heavy chain variable region of humanized antibody 2107 version 5B (VH5B).

(SEQ ID NO: 142)

QVTLKESGPVLVKPTETLTLTCTVSGFSLTSQGVSWVRQPPGKALEWIAA
ISSGGSTYYNSAFKSRLTISRDTSKSQVVLTMTNMDPVDTATYYCARHRP
FYFDYWGQGTLVTVSS

Amino acid sequence of the mature heavy chain variable region of humanized antibody 2107 version 6 (VH6).

(SEQ ID NO: 72)

QVTLKESGPVLVKPTETLTLTCTVSGFSLTSNAVSWVRQPPGKALEWIAA
ISSGGSTYYNSAFKSRLTISRDTSKSQVVLTMTNMDPVDTATYYCARHRP
FYFDYWGQGTLVTVSS

Amino acid sequence of the mature light chain variable region of antibody 2107.4.10.18.

(SEQ ID NO: 73)

DIQVTQSPSLLSASVGDRVTLNCKGSQNIYKSLAWFRLKRGEAPKLLIYD
ANSLQTGIPSRFSGSGSGTDFTLTITSLQPEDVATYFCQQYYSGYTFGAG
TKLELK

Amino acid sequence of the mature light chain variable region of humanized antibody 2107 version 1 (VL1).

(SEQ ID NO: 74)

DIQMTQSPSSLSASVGDRVTITCKGSQNIYKSLAWFQQKPGKVPKLLIYD
ANSLQTGIPSRFSGSGSGTDFTLTISSLQPEDVATYYCQQYYSGYTFGGG
TKVEIK

Amino acid sequence of the mature light chain variable region of humanized antibody 2107 version 2 (VL2).

(SEQ ID NO: 75)

DIQMTQSPSSLSASVGDRVTITCKGSQNIYKSLAWFQLKPGKVPKLLIYD
ANSLQTGIPSRFSGSGSGTDFTLTISSLQPEDVATYYCQQYYSGYTFGGG
TKVEIK

Amino acid sequence of the mature light chain variable region of humanized antibody 2107 version 3 (VL3).

(SEQ ID NO: 76)

DIQMTQSPSSLSASVGDRVTINCKGSQNIYKSLAWFQQKPGKVPKLLIYD
ANSLQTGIPSRFSGSGSGTDFTLTISSLQPEDVATYYCQQYYSGYTFGGG
TKVEIK

Amino acid sequence of the mature heavy chain variable region of antibody 2120.4.19.6.

(SEQ ID NO: 77)

QVQLKESGPGLVQPSQTLSLTCTVSGFSLTSNGVSWVRQPPGKGLEWIAA
ISSGGTTYYNSAFKSRLSISRNTSKSQVLLKMNSLQTEDTAMYFCARRYG
YGWYFDFWGPGTMVTVSS

Amino acid sequence of the mature heavy chain variable region of humanized antibody 2120 version 1 (VH1).

(SEQ ID NO: 78)

QVTLKESGPVLVKPTETLTLTCTVSGFSLTSNGVSWVRQPPGKALEWIAA
ISSGGTTYYNSAFKSRLTISRDTSKSQVVLTMTNMDPVDTATYYCARRYG
YGWYFDFWGQGTLVTVSS

Amino acid sequence of the mature heavy chain variable region of humanized antibody 2120 version 2 (VH2).

(SEQ ID NO: 79)

QVTLKESGPVLVKPTETLTLTCTVSGFSLTSNGVSWVRQPPGKALEWIAA
ISSGGTTYYNSAFKSRLSISRDTSKSQVVLTMTNMDPVDTATYYCARRYG
YGWYFDFWGQGTLVTVSS

Amino acid sequence of the mature heavy chain variable region of humanized antibody 2120 version 3 (VH3).

(SEQ ID NO: 80)

QVTLKESGPVLVKPTETLTLTCTVSGFSLTSSGVSWVRQPPGKALEWIAA
ISSGGTTYYNSAFKSRLTISRDTSKSQVVLTMTNMDPVDTATYYCARRYG
YGWYFDFWGQGTLVTVSS

Amino acid sequence of the mature heavy chain variable region of humanized antibody 2120 version 4 (VH4).

(SEQ ID NO: 81)

QVTLKESGPVLVKPTETLTLTCTVSGFSLTSQGVSWVRQPPGKALEWIAA
ISSGGTTYYNSAFKSRLTISRDTSKSQVVLTMTNMDPVDTATYYCARRYG
YGWYFDFWGQGTLVTVSS

Amino acid sequence of the mature heavy chain variable region of humanized antibody 2120 version 5 (VH5).

(SEQ ID NO: 82)

QVTLKESGPVLVKPTETLTLTCTVSGFSLTSNAVSWVRQPPGKALEWIAA
ISSGGTTYYNSAFKSRLTISRDTSKSQVVLTMTNMDPVDTATYYCARRYG
YGWYFDFWGQGTLVTVSS

Amino acid sequence of the mature light chain variable region of antibody 2120.4.19.6.

(SEQ ID NO: 83)

DIRMTQSPSLLSASVGDRVTLNCKASQNIYNSLAWYQQKLGEGPKVLIPN
ANSLQTGIPSRFSGSGSGTDFTLTISSLQPEDFATYFCQQFYSGYTFGAG
TKLELK

Amino acid sequence of the mature light chain variable region of humanized antibody 2120 version 1 (VL1).

(SEQ ID NO: 84)

DIQMTQSPSSLSASVGDRVTITCKASQNIYNSLAWYQQKPGKAPKVLIFN
ANSLQTGIPSRFSGSGSGTDFTLTISSLQPEDFATYYCQQFYSGYTFGQG
TKLEIK

Amino acid sequence of the mature light chain variable region of humanized antibody 2120 version 2 (VL2).

(SEQ ID NO: 85)

DIQMTQSPSSLSASVGDRVTITCKASQNIYNSLAWYQQKPGKAPKVLIFN
ANSLQTGVPSRFSGSGSGTDFTLTISSLQPEDFATYYCQQFYSGYTFGQG
TKLEIK

Amino acid sequence of the mature light chain variable region of humanized antibody 2120 version 3 (VL3).

(SEQ ID NO: 86)

DIQMTQSPSSLSASVGDRVTINCKASQNIYNSLAWYQQKPGKAPKVLIFN
ANSLQTGIPSRFSGSGSGTDFTLTISSLQPEDFATYYCQQFYSGYTFGQG
TKLEIK

Amino acid sequence of CDRH1 of humanized antibody 2120 version 3 (VH3).

(SEQ ID NO: 87)

GFSLTSQGVS

Amino acid sequence of CDRH1 of humanized antibody 2120 version 4 (VH4).

(SEQ ID NO: 88)

GFSLTSQGVS

Amino acid sequence of CDRH1 of humanized antibody 2120 version 5 (VH5).

(SEQ ID NO: 89)

GFSLTSNAVS

A preferred version of the humanized 2120 antibody is version VH5/VL3 comprising a mature heavy chain variable region having the amino acid sequence designated SEQ ID NO:82 and a mature light chain variable region having the amino acid sequence designated SEQ ID NO:86.

Amino acid sequence of CDRH1 of humanized antibody 2107 version 1 (VH1).

(SEQ ID NO: 90)

SNGVS

Amino acid sequence of CDRH1 of humanized antibody 2107 version 4 (VH4).

(SEQ ID NO: 91)

SSGVS

Amino acid sequence of CDRH3 of humanized antibody 2120 version 1-5 (VH1-VH5).

(SEQ ID NO: 92)

RYGYGWYFDF

A preferred version of the humanized 1749 antibody is version VH3/VL3 comprising a heavy chain having the amino acid sequence designated SEQ ID NO:93 and a light chain having the amino acid sequence designated SEQ ID NO:94.

Amino acid sequence of the mature heavy chain variable region of humanized antibody 1749 version 3 (VH3).

(SEQ ID NO: 93)

EVKLVESGGGLVQPGGSLRLSCAASGFTFSSYIMSWVRQTPEKRLEWVAT
ISSGGSSTYYPDSVKGRFTISRDNAKNTLYLQMSSLKSEDTAMYYCTRDD
DYDVKVFAYWGQGTLVTVSS

Amino acid sequence of the mature light chain variable region of humanized antibody 1749 version 3 (VL3).

(SEQ ID NO: 94)

DIVMTQSPSSLAVSLGERVTINCKSSRSLLNSRIRKNYLAWYQQKPGQSP
KLLIYWASTRESGVPDRFSGSGSGTDFTLTISSLQAEDVAVYYCKQSYNL
LTFGQGTKVEIKR

A preferred version of the humanized 2120 antibody is version VH5 Q1E/VL3 comprising a heavy chain having the amino acid sequence designated SEQ ID NO:100 and a light chain having the amino acid sequence designated SEQ ID NO:86.

Amino acid sequence of the mature heavy chain variable region of antibody 2120.4.19.Q1E, wherein position 1 (Kabat numbering) is occupied by E.

(SEQ ID NO: 95)

EVQLKESGPGLVQPSQTLSLTCTVSGFSLTSNGVSWVRQPPGKGLEWIAA
ISSGGTTYYNSAFKSRLSISRNTSKSQVLLKMNSLQTEDTAMYFCARRYG
YGWYFDFWGPGTMVTVSS

Amino acid sequence of the mature heavy chain variable region of humanized antibody 2120 version 1 Q1E (VH1.Q1E), wherein position 1 (Kabat numbering) is occupied by E.

(SEQ ID NO: 96)

EVTLKESGPVLVKPTETLTLTCTVSGFSLTSNGVSWVRQPPGKALEWIA
AISSGGTTYYNSAFKSRLTISRDTSKSQVVLTMTNMDPVDTATYYCARR
YGYGWYFDFWGQGTLVTVSS

Amino acid sequence of the mature heavy chain variable region of humanized antibody 2120 version 2 Q1E (VH2.Q1E), wherein position 1 (Kabat numbering) is occupied by E.

(SEQ ID NO: 97)

EVTLKESGPVLVKPTETLTLTCTVSGFSLTSNGVSWVRQPPGKALEWIA
AISSGGTTYYNSAFKSRLSISRDTSKSQVVLTMTNMDPVDTATYYCARR
YGYGWYFDFWGQGTLVTVSS

Amino acid sequence of the mature heavy chain variable region of humanized antibody 2120 version 3 Q1E (VH3.Q1E), wherein position 1 (Kabat numbering) is occupied by E.

(SEQ ID NO: 98)

EVTLKESGPVLVKPTETLTLTCTVSGFSLTSSGVSWVRQPPGKALEWIA
AISSGGTTYYNSAFKSRLTISRDTSKSQVVLTMTNMDPVDTATYYCARR
YGYGWYFDFWGQGTLVTVSS

Amino acid sequence of the mature heavy chain variable region of humanized antibody 2120 version 4 Q1E (VH4.Q1E), wherein position 1 (Kabat numbering) is occupied by E.

(SEQ ID NO: 99)

EVTLKESGPVLVKPTETLTLTCTVSGFSLTSQGVSWVRQPPGKALEWIA
AISSGGTTYYNSAFKSRLTISRDTSKSQVVLTMTNMDPVDTATYYCARR
YGYGWYFDFWGQGTLVTVSS

Amino acid sequence of the mature heavy chain variable region of humanized antibody 2120 version 5 Q1E (VH5.Q1E), wherein position 1 (Kabat numbering) is occupied by E.

(SEQ ID NO: 100)

EVTLKESGPVLVKPTETLTLTCTVSGFSLTSNAVSWVRQPPGKALEWIA
AISSGGTTYYNSAFKSRLTISRDTSKSQVVLTMTNMDPVDTATYYCARR
YGYGWYFDFWGQGTLVTVSS

Amino acid sequence of a humanized 2120 light chain constant region, with Arginine at the N-terminus.

(SEQ ID NO: 101)

RTVAAPSVFIFPPSDEQLKSGTASVVCLLNNFYPREAKVQWKVDNALQS
GNSQESVTEQDSKDSTYSLSSTLTLSKADYEKHKVYACEVTHQGLSSPV
TKSFNRGEC

Amino acid sequence of a humanized 2120 light chain constant region, without Arginine at the N-terminus.

(SEQ ID NO: 102)

TVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVT
KSFNRGEC

Amino acid sequence of a humanized 2120 heavy chain constant region.

(SEQ ID NO: 103)

ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPELLGGPSVFLFPPKPKDTLMISRTPEVTCVVV
DVSHEDPEVKFNWYVDGVEVHNVKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

Amino acid sequence of a BIP version heavy chain G1m3 allotype constant region.

(SEQ ID NO: 104)

ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPELLGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

Amino acid sequence of a BIP version heavy chain G1m3 allotype constant region.

(SEQ ID NO: 105)

ASTKGPSVFPLAPSSKSTSGGTAALGCLVKDYFPEPVTVSWNSGALTSG
VHTFPAVLQSSGLYSLSSVVTVPSSSLGTQTYICNVNHKPSNTKVDKRV
EPKSCDKTHTCPPCPAPEAAGGPSVFLFPPKPKDTLMISRTPEVTCVVV
DVSHEDPEVKFNWYVDGVEVHNAKTKPREEQYNSTYRVVSVLTVLHQDW
LNGKEYKCKVSNKALPAPIEKTISKAKGQPREPQVYTLPPSREEMTKNQ
VSLTCLVKGFYPSDIAVEWESNGQPENNYKTTPPVLDSDGSFFLYSKLT
VDKSRWQQGNVFSCSVMHEALHNHYTQKSLSLSPGK

Amino acid sequence of a mature light chain region of humanized antibody 2120 version 3 (VL3+light chain constant region).

(SEQ ID NO: 106)

DIQMTQSPSSLSASVGDRVTINCKASQNIYNSLAWYQQKPGKAPKVLIF
NANSLQTGIPSRFSGSGSGTDFTLTISSLQPEDFATYYCQQFYSGYTFG
QGTKLEIKRTVAAPSVFIFPPSDEQLKSGTASVVCLLNNFYPREAKVQW
KVDNALQSGNSQESVTEQDSKDSTYSLSSTLTLSKADYEKHKVYACEVT
HQGLSSPVTKSFNRGEC

Amino acid sequence of a mature heavy chain region of humanized antibody 2120 version 5 (VH5+BIP version heavy chain G1m3 allotype constant region).

(SEQ ID NO: 107)

QVTLKESGPVLVKPTETLTLTCTVSGFSLTSNAVSWVRQPPGKALEWIA
AISSGGTTYYNSAFKSRLTISRDTSKSQVVLTMTNMDPVDTATYYCARR
YGYGWYFDFWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK

Amino acid sequence of a mature heavy chain region of humanized antibody 2120 version 5 (VH5+BIP version heavy chain G1m3 allotype constant region).

(SEQ ID NO: 108)

QVTLKESGPVLVKPTETLTLTCTVSGFSLTSNAVSWVRQPPGKALEWIA
AISSGGTTYYNSAFKSRLTISRDTSKSQVVLTMTNMDPVDTATYYCARR
YGYGWYFDFWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPKSCDKTHTCPPCPAPEAAGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK

Amino acid sequence of a mature heavy chain region of humanized antibody 2120 version 5 Q1E (VH5.Q1E+BIP version heavy chain G1m3 allotype constant region).

(SEQ ID NO: 109)

EVTLKESGPVLVKPTETLTLTCTVSGFSLTSNAVSWVRQPPGKALEWIA
AISSGGTTYYNSAFKSRLTISRDTSKSQVVLTMTNMDPVDTATYYCARR
YGYGWYFDFWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPKSCDKTHTCPPCPAPELLGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK

Amino acid sequence of a mature heavy chain region of humanized antibody 2120 version 5 Q1E (VH5.Q1E+BIP version heavy chain G1m3 allotype constant region).

(SEQ ID NO: 110)

EVTLKESGPVLVKPTETLTLTCTVSGFSLTSNAVSWVRQPPGKALEWIA
AISSGGTTYYNSAFKSRLTISRDTSKSQVVLTMTNMDPVDTATYYCARR
YGYGWYFDFWGQGTLVTVSSASTKGPSVFPLAPSSKSTSGGTAALGCLV
KDYFPEPVTVSWNSGALTSGVHTFPAVLQSSGLYSLSSVVTVPSSSLGT
QTYICNVNHKPSNTKVDKRVEPKSCDKTHTCPPCPAPEAAGGPSVFLFP
PKPKDTLMISRTPEVTCVVVDVSHEDPEVKFNWYVDGVEVHNAKTKPRE
EQYNSTYRVVSVLTVLHQDWLNGKEYKCKVSNKALPAPIEKTISKAKGQ
PREPQVYTLPPSREEMTKNQVSLTCLVKGFYPSDIAVEWESNGQPENNY
KTTPPVLDSDGSFFLYSKLTVDKSRWQQGNVFSCSVMHEALHNHYTQKS
LSLSPGK

Amino acid sequence of a mature light chain region of humanized antibody 1749 version 3 (VL3+light chain constant region).

(SEQ ID NO: 111)

DIVMTQSPSSLAVSLGERVTINCKSSRSLLNSRIRKNYLAWYQQKPGQSP
KLLIYWASTRESGVPDRFSGSGSGTDFTLTISSLQAEDVAVYYCKQSYNL
LTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

Amino acid sequence of a mature heavy chain region of humanized antibody 1749 version 3 (VH3+BIP version heavy chain G1m3 allotype constant region).

(SEQ ID NO: 112)

EVKLVESGGGLVQPGGSLRLSCAASGFTFSSYIMSWVRQTPEKRLEWVAT
ISSGGSSTYYPDSVKGRFTISRDNAKNTLYLQMSSLKSEDTAMYYCTRDD
DYDVKVFAYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK

Amino acid sequence of a mature heavy chain region of humanized antibody 1749 version 3 (VH3+BIP version heavy chain G1m3 allotype constant region).

(SEQ ID NO: 113)

EVKLVESGGGLVQPGGSLRLSCAASGFTFSSYIMSWVRQTPEKRLEWVAT
ISSGGSSTYYPDSVKGRFTISRDNAKNTLYLQMSSLKSEDTAMYYCTRDD
DYDVKVFAYWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKRVEPKSCDKTHTCPPCPAPEAAGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSREEMTKNQVSLTCLVKGFYPSDIAVEWESNGQPENNYKTTPPV
LDSDGSFFLYSKLTVDKSRWQQGNVFSCSVMHEALHNHYTQKSLSLSPGK

Some exemplary antibodies binding to laminin alpha-4 and inhibiting its interaction with MCAM are characterized by the following sequences.

Amino acid sequence of humanized 19C12 heavy chain variable region version 1 (H1).

(SEQ ID NO: 114)

QVQLQQSGAELVKPGASVKISCKASGYAFSTYWMNWVKQAPGEGLEWIGQ
IYPGDGDTNYNGKFKGRVTLTADKSTSTAYMELSSLRSEDTAVYFCARSD
GYYDYWGQGTTVTVSS

Amino acid sequence of humanized 19C12 heavy chain variable region version 2 (H2).

(SEQ ID NO: 115)

QVQLQQSGAELVKPGASVKVSCKASGYAFSTYWMNWVRQAPGQGLEWIGQ
IYPGDGDTNYNGKFKGRVTITADKSTSTAYMELSSLRSEDTAVYFCARSD
GYYDYWGQGTTVTVSS

Amino acid sequence of humanized 19C12 heavy chain variable region version 3 (H3).

(SEQ ID NO: 116)

EVQLQQSGAELVKPGASVKVSCKASGYAFSTYWMNWVRQAPGQGLEWIGQ
IYPGDGDTNYNGKFKGRVTITADKSTSTAYMELSSLRSEDTAVYFCARSD
GYYDYWGQGTTVTVSS

Amino acid sequence of humanized 19C12 light chain variable region version 1 (L1).

(SEQ ID NO: 117)

NIVLTQSPASLAVSLGERATISCRASESVDSYGTSFMHWYQQKPGQPPKL
LICLASSLESGVPDRFSGSGSRTDFTLTISSLQAEDVATYYCQQNNEDPP
TFGQGTKLEIKR

Amino acid sequence of humanized 19C12 light chain variable region version 2 (L2).

(SEQ ID NO: 118)

NIVLTQSPASLAVSLGERATISCRASESVDSYGTSFMHWYQQKPGQPPKL
LISLASSLESGVPDRFSGSGSGTDFTLTISSLQAEDVATYYCQQNNEDPP
TFGQGTKLEIKR

Amino acid sequence of humanized 19C12 light chain variable region version 3 (L3).

(SEQ ID NO: 119)

NIVLTQSPASLAVSLGERATISCRASESVDSYGTSFMHWYQQKPGQPPKL
LICLASSLESGVPARFSGSGSRTDFTLTIDPVEAEDAATYYCQQNNEDPP
TFGAGTKLEIKR

Amino acid sequence of humanized 19C12 light chain variable region version 4 (L4).

(SEQ ID NO: 120)

NIVLTQSPASLAVSLGERATISCRASESVDSYGTSFMHWYQQKPGQPPKL
LISLASSLESGVPARFSGSGSGTDFTLTISPVEAEDAATYYCQQNNEDPP
TFGAGTKLEIKR

Amino acid sequence of humanized 19C12 light chain variable region version 5 (L5).

(SEQ ID NO: 121)

DIVLTQSPASLAVSLGERATISCRASESVDSYGTSFMHWYQQKPGQPPKL
LISLASSLESGVPARFSGSGSGTDFTLTISPLQAEDVATYYCQQNNEDPP
TFGQGTKLEIKR

Amino acid sequence of humanized 19C12 light chain variable region version 6 (L6).

(SEQ ID NO: 122)

DIVLTQSPASLAVSLGERATISCRASESVDSYGTSFMHWYQQKPGQPPKL
LISLASSLESGVPARFSGSGSGTDFTLTISPVEAEDAATYYCQQNNEDPP
TFGAGTKLEIKR

Amino acid sequence of exemplary mature heavy chain of 19C12 humanized antibody (H1+BIP version heavy chain G1m3 allotype constant region).

(SEQ ID NO: 123)

QVQLQQSGAELVKPGASVKISCKASGYAFSTYWMNWVKQAPGEGLEWIGQ
IYPGDGDTNYNGKFKGRVTLTADKSTSTAYMELSSLRSEDTAVYFCARSD
GYYDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

Amino acid sequence of exemplary mature heavy chain of 19C12 humanized antibody (H1+BIP version heavy chain G1m3 allotype constant region).

(SEQ ID NO: 124)

QVQLQQSGAELVKPGASVKISCKASGYAFSTYWMNWVKQAPGEGLEWIGQ
IYPGDGDTNYNGKFKGRVTLTADKSTSTAYMELSSLRSEDTAVYFCARSD
GYYDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPEAAGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

Amino acid sequence of exemplary mature heavy chain of 19C12 humanized antibody (H2+BIP version heavy chain G1m3 allotype constant region).

(SEQ ID NO: 125)

QVQLQQSGAELVKPGASVKVSCKASGYAFSTYWMNWVRQAPGQGLEWIGQ
IYPGDGDTNYNGKFKGRVTITADKSTSTAYMELSSLRSEDTAVYFCARSD
GYYDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

Amino acid sequence of exemplary mature heavy chain of 19C12 humanized antibody (H2+BIP version heavy chain G1m3 allotype constant region).

(SEQ ID NO: 126)

QVQLQQSGAELVKPGASVKVSCKASGYAFSTYWMNWVRQAPGQGLEWIGQ
IYPGDGDTNYNGKFKGRVTITADKSTSTAYMELSSLRSEDTAVYFCARSD
GYYDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPEAAGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

Amino acid sequence of exemplary mature heavy chain of 19C12 humanized antibody (H3+BIP version heavy chain G1m3 allotype constant region).

(SEQ ID NO: 127)

EVQLQQSGAELVKPGASVKVSCKASGYAFSTYWMNWVRQAPGQGLEWIGQ
IYPGDGDTNYNGKFKGRVTITADKSTSTAYMELSSLRSEDTAVYFCARSD
GYYDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

Amino acid sequence of exemplary mature heavy chain of 19C12 humanized antibody (H3+BIP version heavy chain G1m3 allotype constant region).

(SEQ ID NO: 128)

EVQLQQSGAELVKPGASVKVSCKASGYAFSTYWMNWVRQAPGQGLEWIGQ
IYPGDGDTNYNGKFKGRVTITADKSTSTAYMELSSLRSEDTAVYFCARSD
GYYDYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKRVEPKSCDKTHTCPPCPAPEAAGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSREEMTKNQVSLTCLVKGFYPSDIAVEWESNGQPENNYKTTPPVLDSD
GSFFLYSKLTVDKSRWQQGNVFSCSVMHEALHNHYTQKSLSLSPGK

Amino acid sequence of exemplary mature light chain of 19C12 humanized antibody (L1+light chain constant region with arginine at N-terminus).

(SEQ ID NO: 129)

NIVLTQSPASLAVSLGERATISCRASESVDSYGTSFMHWYQQKPGQPPKL
LICLASSLESGVPDRFSGSGSRTDFTLTISSLQAEDVATYYCQQNNEDPP
TFGQGTKLEIKRRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

Amino acid sequence of exemplary mature light chain of 19C12 humanized antibody (L1+light chain constant region without arginine at N-terminus).

(SEQ ID NO: 130)

NIVLTQSPASLAVSLGERATISCRASESVDSYGTSFMHWYQQKPGQPPKL
LICLASSLESGVPDRFSGSGSRTDFTLTISSLQAEDVATYYCQQNNEDPP
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

Amino acid sequence of exemplary mature light chain of 19C12 humanized antibody (L2+light chain constant region with arginine at N-terminus).

(SEQ ID NO: 131)

NIVLTQSPASLAVSLGERATISCRASESVDSYGTSFMHWYQQKPGQPPKL
LISLASSLESGVPDRFSGSGSGTDFTLTISSLQAEDVATYYCQQNNEDPP
TFGQGTKLEIKRRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

Amino acid sequence of exemplary mature light chain of 19C12 humanized antibody (L2+light chain constant region without arginine at N-terminus).

(SEQ ID NO: 132)

NIVLTQSPASLAVSLGERATISCRASESVDSYGTSFMHWYQQKPGQPPKL
LISLASSLESGVPDRFSGSGSGTDFTLTISSLQAEDVATYYCQQNNEDPP
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

Amino acid sequence of exemplary mature light chain of 19C12 humanized antibody (L3+light chain constant region with arginine at N-terminus).

(SEQ ID NO: 133)

NIVLTQSPASLAVSLGERATISCRASESVDSYGTSFMHWYQQKPGQPPKL
LICLASSLESGVPARFSGSGSRTDFTLTIDPVEAEDAATYYCQQNNEDPP
TFGAGTKLEIKRRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

Amino acid sequence of exemplary mature light chain of 19C12 humanized antibody (L3+light chain constant region without arginine at N-terminus).

(SEQ ID NO: 134)

NIVLTQSPASLAVSLGERATISCRASESVDSYGTSFMHWYQQKPGQPPKL
LICLASSLESGVPARFSGSGSRTDFTLTIDPVEAEDAATYYCQQNNEDPP
TFGAGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

Amino acid sequence of exemplary mature light chain of 19C12 humanized antibody (L4+light chain constant region with arginine at N-terminus).

(SEQ ID NO: 135)

NIVLTQSPASLAVSLGERATISCRASESVDSYGTSFMHWYQQKPGQPPKL
LISLASSLESGVPARFSGSGSGTDFTLTISPVEAEDAATYYCQQNNEDPP
TFGAGTKLEIKRRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

Amino acid sequence of exemplary mature light chain of 19C12 humanized antibody (L4+light chain constant region without arginine at N-terminus).

(SEQ ID NO: 136)

NIVLTQSPASLAVSLGERATISCRASESVDSYGTSFMHWYQQKPGQPPKL
LISLASSLESGVPARFSGSGSGTDFTLTISPVEAEDAATYYCQQNNEDPP
TFGAGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

Amino acid sequence of exemplary mature light chain of 19C12 humanized antibody (L5+light chain constant region with arginine at N-terminus).

(SEQ ID NO: 137)

DIVLTQSPASLAVSLGERATISCRASESVDSYGTSFMHWYQQKPGQPPKL
LISLASSLESGVPARFSGSGSGTDFTLTISPLQAEDVATYYCQQNNEDPP
TFGQGTKLEIKRRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

Amino acid sequence of exemplary mature light chain of 19C12 humanized antibody (L5+light chain constant region without arginine at N-terminus).

(SEQ ID NO: 138)

DIVLTQSPASLAVSLGERATISCRASESVDSYGTSFMHWYQQKPGQPPKL
LISLASSLESGVPARFSGSGSGTDFTLTISPLQAEDVATYYCQQNNEDPP
TFGQGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

Amino acid sequence of exemplary mature light chain of 19C12 humanized antibody (L6+light chain constant region with arginine at N-terminus).

(SEQ ID NO: 139)

DIVLTQSPASLAVSLGERATISCRASESVDSYGTSFMHWYQQKPGQPPKL
LISLASSLESGVPARFSGSGSGTDFTLTISPVEAEDAATYYCQQNNEDPP
TFGAGTKLEIKRRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC

Amino acid sequence of exemplary mature light chain of 19C12 humanized antibody (L6+light chain constant region without arginine at N-terminus).

(SEQ ID NO: 140)

DIVLTQSPASLAVSLGERATISCRASESVDSYGTSFMHWYQQKPGQPPKL
LISLASSLESGVPARFSGSGSGTDFTLTISPVEAEDAATYYCQQNNEDPP
TFGAGTKLEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC

Exemplary antibodies binding to laminin alpha-4 and inhibiting its interaction with MCAM include any permutations or combinations of the exemplified humanized 19C12 mature heavy and light chain variable regions (e.g., H1L1, H1L2, H1L3, H1L4, H1L5, H1L6, H2L1, H2L2, H2L3, H2L4, H2L5, H2L6, H3L1, H3L2, H3L3, H3L4, H3L5, and H3L6). Preferred versions of humanized 19C12 antibodies are versions comprising H2 or H3 and/or L3 or L6, such as a combination of H3 and L6 or a combination of H2 and L3.

The heavy and light chain variable regions of chimeric, veneered or humanized antibodies can be linked to at least a portion of a human constant region. The choice of constant region depends, in part, whether antibody-dependent cell-mediated cytotoxicity, antibody dependent cellular phagocytosis and/or complement dependent cytotoxicity are desired. For example, human isotopes IgG1 and IgG3 have complement-dependent cytotoxicity and human isotypes IgG2 and IgG4 do not. Human IgG1 and IgG3 also induce stronger cell mediated effector functions than human IgG2 and IgG4. Light chain constant regions can be lambda or kappa.

One or several amino acids at the amino or carboxy terminus of the light and/or heavy chain, such as the C-terminal lysine of the heavy chain, may be missing or derivatized in a proportion or all of the molecules. Substitutions can be made in the constant regions to reduce or increase effector function such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006), or to prolong half-life in humans (see, e.g., Hinton et al., J. Biol. Chem. 279:6213, 2004). Exemplary substitutions include a Gln at position 250 and/or a Leu at position 428 (EU numbering is used in this paragraph for the constant region) for increasing the half-life of an antibody. Substitution at any or all of positions 234, 235, 236 and/or 237 reduces affinity for Fcγ receptors, particularly FcγRI receptor (see, e.g., U.S. Pat. No. 6,624,821). An alanine substitution at positions 234, 235, and 237 of human IgG1 can be used for reducing effector functions. Some antibodies have alanine substitution at positions 234, 235 and 237 of human IgG1 for reducing effector functions. Optionally, positions 234, 236 and/or 237 in human IgG2 are substituted with alanine and position 235 with glutamine (see, e.g., U.S. Pat. No. 5,624,821). In some antibodies, a mutation at one or more of positions 241, 264, 265, 270, 296, 297, 322, 329, and 331 by EU numbering of human IgG1 is used. In some antibodies, a mutation at one or more of positions 318, 320, and 322 by EU numbering of human IgG1 is used. In some antibodies, positions 234 and/or 235 are substituted with alanine and/or position 329 is substituted with glycine. In some antibodies, positions 234 and 235 are substituted with alanine, such as in SEQ ID NO:105. In some antibodies, the isotype is human IgG2 or IgG4. An exemplary human light chain kappa constant region has the amino acid sequence of SEQ ID NO:101. The N-terminal arginine of SEQ ID NO:101 can be omitted, in which case light chain kappa constant region has the amino acid sequence of SEQ ID NO:102. An exemplary human IgG1 heavy chain constant region has the amino acid sequence of SEQ ID NO:103 (with or without the C-terminal lysine). Antibodies can be expressed as tetramers containing two light and two heavy chains, as separate heavy chains, light chains, as Fab, Fab′, F(ab′)2, and Fv, or as single chain antibodies in which heavy and light chain mature variable domains are linked through a spacer.

Human constant regions show allotypic variation and isoallotypic variation between different individuals, that is, the constant regions can differ in different individuals at one or more polymorphic positions. Isoallotypes differ from allotypes in that sera recognizing an isoallotype bind to a non-polymorphic region of one or more other isotypes. Thus, for example, another heavy chain constant region is of IgG1 G1m3 allotype and has the amino acid sequence of SEQ ID NO:104. Another heavy chain constant region has the amino acid sequence of SEQ ID NO:104 except that it lacks the C-terminal lysine. Another heavy chain constant region has the amino acid sequence of SEQ ID NO:105. Yet another heavy chain constant region has the amino acid sequence of SEQ ID NO:105 except that it lacks the C-terminal lysine.

The invention further provides nucleic acids encoding any of the above constant regions. Optionally, such nucleic acids further encode a signal peptide and can be expressed with the signal peptide linked to the constant region.

MCAM antagonists can be screened as follows. MCAM-expressing cells comprising the steps of: (a) incubating a population of cells expressing a laminin α4 chain, e.g., an α4 chain of laminin 411, with MCAM, in the presence or absence of a candidate molecule; (b) monitoring the level of binding of MCAM to the cells; and (c) identifying said candidate molecule as an inhibitor of CNS infiltration by MCAM-expressing cells if the level of MCAM binding is lower in the presence than in the absence of said candidate molecule. An alternate screening protocol involves the use of a population of cells expressing a laminin α4 chain, e.g., an α4 chain of laminin 411, which can be incubated with MCAM, in the presence and absence of a test compound, and binding of MCAM to the cell population monitored, e.g. by fluorescent microscopy.

### IV. Multiple Sclerosis and Other Autoimmune Diseases

The present methods can be used to treat multiple sclerosis in any or all of its subtypes. At least four subtypes exist. Relapsing-remitting MS (RR-MS) is the most common form of MS and is characterized by clearly defined exacerbations/relapses (acute attacks) followed by partial or complete recovery. There is no disease progression between the relapse periods. Initially (at the time of diagnosis) RR-MS represents about 85% of all newly diagnosed subjects. The definition of relapse requires the new symptom or sign to be present for at least 24 hours, to not be associated with a fever or intercurrent illness (such as the “flu” or a urinary tract infection), because an elevated body temperature can unmask silent or old lesions.

Primary progressive (PP-MS) is continuous from the beginning without clear relapses. There can be plateaus (periods of stabilization). 10-15% of all MS subjects are in this group and it tends to occur in older aged individuals. The female to male ratio is equal in this group, unlike other forms where females predominant by about 2:1. Also PP-MS tends to present with fewer cerebral MRI changes and more myelopathy/spinal cord related changes.

Secondary progressive form (SP-MS) starts as a RR-MS and later steady progression occurs with or without relapses. Approximately 50% of relapsing-remitting subjects progress to the secondary progressive form.

Progressive Relapsing form (PR-MS), occurring in about 5% of individuals, is progressive from the onset with superimposed relapses (with or without recovery).

Diagnosis of MS is usually based on a medical history, a neurologic exam and various tests, including magnetic resonance imaging (MRI), evoked potentials (EP) and spinal fluid analysis. A definitive diagnosis of MS requires evidence of damage in at least two separate areas of the central nervous system (CNS), which includes the brain, spinal cord and optic nerves and evidence that the damage occurred at least one month apart and exclusion of all other possible diagnoses. As well as therapeutically treating subjects having a diagnosis of MS by art-recognized criteria, the present methods can also be used prophylactically to treat individually having at least one sign or symptom of MS placing them at increased risk of progression to MS compared with the general population of healthy individuals. For example, the methods can be used to treat individuals who have had one attack (also called a relapse or an exacerbation) of MS-like symptoms—referred to as a clinically-isolated syndrome (CIS), who may or may not go on to develop MS. Individuals at risk of developing MS can also be identified by presence of an antibody to the protein KIR4.1 in their serum, among other methods.

The combined treatment methods can also be used against other autoimmune diseases, particularly those, such as Crohn's disease or rheumatoid arthritis in which natalizumab has individually demonstrated evidence of efficacy, as well as other autoimmune diseases in which natalizumab alone has not hitherto show evidence of acceptable efficacy but can do in combination with an MCAM antagonist. Autoimmune diseases include systemic autoimmune diseases, organ- or tissue-specific autoimmune diseases, and diseases that exhibit autoimmune-type expressions. In these diseases, the body develops a cellular and/or humoral immune response against one of its own antigens, leading to destruction of that antigen and potentially crippling and/or fatal consequences. The cellular response if present can be B-cell or T-cell or both. The present methods are particularly amenable to treating autoimmune diseases mediated at least in part by T-cells, at least some of which are T_{H}1 or T_{H}17 cells, and preferably both T_{H}1 and T_{H}17 cells. T_{H}17 cells, a lineage T helper cells characterized by production of interleukin (IL)-17 and IL-22, have been reported to enter tissues to facilitate pathogenic autoimmune responses, including multiple sclerosis in humans and experimental autoimmune encephalomyelitis (EAE) in mice. See, e.g., Cua et al., Nature 421: 744-748 (2003); Ivonov et al., Cell 126: 1121-1133 (2006). TH17 cells may initiate or propagate an inflammatory response by their specific recruitment to and infiltration of tissue. Examples of T-cell mediated autoimmune diseases amenable to treatment include multiple sclerosis, Crohn's Disease, rheumatoid arthritis, psoriasis, psoriatic arthritis, and sarcoidosis.

Examples of additional autoimmune diseases amenable to treatment include Graves' disease, Hashimoto's thyroiditis, autoimmune polyglandular syndrome, insulin-dependent diabetes mellitus (type 1 diabetes), insulin-resistant diabetes mellitus (type 2 diabetes), immune-mediated infertility, autoimmune Addison's disease, pemphigus vulgaris, pemphigus foliaceus, dermatitis herpetiformis, autoimmune alopecia, vitiligo, autoimmune hemolytic anemia, idiopathic thrombocytopenic purpura, autoimmune thrombocytopenic purpura, pernicious anemia, myasthenia gravis, Guillain-Barre syndrome, stiff man syndrome, acute rheumatic fever, sympathetic ophthalmia, Goodpasture's syndrome, autoimmune uveitis, temporal arteritis, Bechet's disease, inflammatory bowel diseases, ulcerative colitis, primary biliary cirrhosis, autoimmune hepatitis, autoimmune oophoritis, fibromyalgia, polymyositis, dermatomyositis, ankylosing spondylitis, Takayashu arteritis, panniculitis, pemphigoid, vasculitis of unknown origin, anca negative vasculitis, anca positive vasculitis, systemic lupus erythematosus, scleroderma, systemic necrotizing vasculitis, Wegener's granulomatosis, CREST syndrome, antiphospholipid syndrome, Sjogren's syndrome, eosinophilic gastroenteritis, atypical topical dermatitis, cardiomyopathy, post-infectious syndromes, postinfectious endomyocarditis, and celiac disease.

### V. Combination Treatments

The invention provides methods of treatment in which the indicated alpha-4 integrin and MCAM antagonists are administered to subjects having or at increased risk of multiple sclerosis, or other disease disclosed herein. As further described below, the antagonists can be administered concurrently or sequentially and, if administered sequentially, then in either order. The methods are particularly amenable to treatment of human subjects.

The antagonists can be administered to a subject by any suitable route, especially, in the case of biologics, parentally by intravenous (IV) infusion or bolus injection, intramuscularly or subcutaneously or intraperitoneally. IV infusion can be given over for example, periods from 15 minutes to 3 hours, more typically from 30-90 or 45-75 minutes.

The antagonists are administered in a regime (doses, frequencies of administration, routes of administration, relative order of administration) effective to reduce signs or symptoms of disease or at least slow progression or exacerbation of symptoms in the case of therapeutic treatment and delay or inhibit development of symptoms in the case of prophylactic treatment.

When an alpha-4 integrin antagonist is used in combination with an MCAM antagonist, the combination may take place over any convenient timeframe. For example, each antagonist can be administered to a subject on the same day, and the antagonists can even be administered in the same intravenous infusion. However, the antagonists can also be administered on alternating days or alternating weeks, fortnights or months, and so on. In some methods, the respective antagonists are administered with sufficient proximity in time that the antagonists are simultaneously present (e.g., in the serum) at detectable levels in the subject being treated. In some methods, an entire course of treatment of one antagonist consisting of a number of doses over a time period (see above) is followed by a course of treatment of the other antagonist also consisting of a number of doses. In some methods, treatment with the antagonist administered second is begun if the subject has resistance or develops resistance to the antagonist administered initially. In some methods, treatment of the antagonist administered second is begun if the nature of autoimmune reaction in the patient changes from being primarily mediated by T_{H}1 cells to T_{H}17 cells.

In some methods, a course of treatment of an alpha-4 integrin antagonist is administered first followed by a course of treatment of an MCAM antagonist. In some such methods, the subject has relapsing-remitting multiple sclerosis or enhanced risk thereof on initiating the first alpha-4 integrin antagonist course of treatment, and has transitioned to the secondary progressive form on initiating the course of treatment with the MCAM antagonist.

The subject may receive only a single course of treatment with each antagonist or multiple courses with one or both antagonists. Sometimes a recovery period of one, two or several days or weeks is allowed between administration of the two antagonists if this is beneficial to the subject in the judgment of the attending physician. When a suitable treatment regime has already been established for one of the antagonists (e.g., 300 mg every four weeks by iv infusion for natalizumab), that regime can be used when the antagonist is in used in combination with the other antagonist, although lower dosages can also be administered because of cooperative or synergistic effects between the antagonists. The antagonists can be administered for life or until the disease progresses without apparent inhibition by the antagonists.

The efficacy of combined treatments can be assessed by conventional end points for multiple sclerosis including total number of lesions in the CNS, gadolinium-enhanced (i.e., new lesions), frequency of relapses, time to first relapse, and various disability indices, such as the Expanded Disability Status Scale or Functional System Score. When administering treatment with two antagonists, the regimes with which the respective antagonists are administered are combined in such a manner that each antagonist can make a contribution to the therapy. Preferably, treatment according to the invention with the first and second antagonists leads to an increase in an objective measure of favorable response compared with each of the first and second antagonist individually used at the same molar dose individually as in the combination (the antagonists work cooperatively). More preferably, treatment with the first and second antagonists leads to an increase in an objective measure of favorable response compared with each of the first and second antagonist individually used at the same molar dosage individually as the combined molar dosage in the combination (i.e., the antagonists act synergistically). Preferably, the increase in an objective measure of response for combined antagonists is by at least 10%, 20%, 30% or 40% but preferably 50%, 60% to 70% or even 80%, 90% or 100% compared to treatment with the more effective of the individual antagonists, at an equimolar dose with the same antagonist in the combination or at an equimolar dose with the molar dose of the combined antagonists in the combination.

Typically, objective response rates are evaluated in a clinical trial (e.g., a phase II, phase II/III or phase III trial), from subjects receiving the combined treatment relative to control group of subjects receiving individual treatments or placebo.

The dose of each antibody or other biologic antagonists is sometimes 0.1 to 20 mg/kg body weight, for example 0.5 to 10 or 3-7 mg/kg. Sometimes the dose is 1, 2, 3, 4, 5 or 6 mg/kg, but can be as high as 10 mg/kg or even 15 or 20 or 30 mg/kg. A fixed unit dose may also be given, for example, 50, 100, 200, 500 or 1000 mg, or the dose may be based on the subject's surface area, e.g., 100 mg/m.sup.2. Exemplary fixed doses of each agent are s 50-500 mg/subject, 100-200 mg/subject or 50-150 mg/subject. Doses can be administered daily, biweekly, weekly, every other week, every four weeks, or at some other interval. Effective doses vary depending upon many different factors, including means of administration, target site, physiological state of the subject including type of multiple sclerosis, genetics status of the subject, whether the subject is human or an animal, other medications administered, and whether treatment is prophylactic or therapeutic.

Small molecule antagonists are typically administered more often, preferably once a day, but 2, 3, 4 or more times per day is also possible, as is every two days, weekly or at some other interval. Small molecule drugs are often taken orally but parenteral administration is also possible, e.g., by IV infusion or bolus injection or subcutaneously or intramuscularly. Doses of small molecule drugs are typically 1 or 10 to 1000 mg, with 100, 150, 200 or 250 mg very typical, with the optimal dose established in clinical trials.

The combination of alpha-4 integrin and MCAM antagonists can also be administered with any other drug effective against multiple sclerosis, such as those disclosed in the Background section.

Optionally, an alpha-4 integrin antagonist and MCAM antagonist can be combined in a combination product or kit, for example, as separate vials in the same package, or holder. Some combinations of these antagonists can also be mixed in the same composition. Such compositions and kits can be formed either by a manufacturer or by a health care provider. Kits and compositions can be provided with instructions for use in any of the methods of the invention.

### VI. Pharmaceutical Compositions, Dosages, Routes of Administration

Pharmaceutical compositions for parenteral administration are can be sterile and substantially isotonic (230-350 mOsm/kg) and manufactured under GMP conditions. Pharmaceutical compositions can be provided in unit dosage form (i.e., the dosage for a single administration). Pharmaceutical compositions can be formulated using one or more physiologically acceptable carriers, diluents, excipients or auxiliaries. The formulation depends on the route of administration chosen. For injection, antibodies can be formulated in aqueous solutions, preferably in physiologically compatible buffers such as Hank's solution, Ringer's solution, or physiological saline or acetate buffer (to reduce discomfort at the site of injection). The solution can contain formulatory antagonists such as suspending, stabilizing and/or dispersing antagonists. Alternatively antibodies can be in lyophilized form for constitution with a suitable vehicle, e.g., sterile pyrogen-free water, before use.

All patent filings, websites, other publications, accession numbers and the like cited above or below are incorporated by reference in their entirety for all purposes to the same extent as if each individual item were specifically and individually indicated to be so incorporated by reference. If different versions of a sequence are associated with an accession number at different times, the version associated with the accession number at the effective filing date of this application is meant. The effective filing date means the earlier of the actual filing date or filing date of a priority application referring to the accession number if applicable Likewise if different versions of a publication, website or the like are published at different times, the version most recently published at the effective filing date of the application is meant unless otherwise indicated. Any feature, step, element, embodiment, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### Examples

Although natalizumab's saturation rates should completely block entry of (T) lymphocytes, subjects under natalizumab treatment still have a detectable number of immune cells in the CSF and putatively the CNS, suggesting that there are alternative mechanisms for some immune cell populations to enter the CNS. We therefore investigated the mechanisms of early migration events and its main molecules very late antigen-4 (VLA-4, α4β1=CD49d/CD29 integrin) and P-selectin glycoprotein ligand-1 (PSGL-1=CD162). These molecules and their ligands or counter receptors (VCAM-1 in the case of VLA-4 and P-selectin in the case of PSGL-1) are involved in tethering, rolling, and adhesion of T cells to endothelial barriers and successful extravasation into the CNS.

### Material and Methods:

### Subjects and Healthy Controls

381 subjects with the diagnosis of clinically definite active RRMS according to the 2005 revised McDonald diagnostic criteria (Polman et al., Ann. Neurol. 58(6):840-846 (2005)) were enrolled in this study. These MS subjects had been treated continuously with natalizumab for up to 80 months and were stable by assessment of clinical and MRI parameters. 21 subjects in this cohort underwent analysis of CSF in parallel to assessment of peripheral blood. 39 age and sex-matched healthy donors (HD) with no previous history of neurological or immune-mediated diseases served as controls. Furthermore, 49 natalizumab-naïve MS subjects, 32 subjects with non-inflammatory neurological diseases (NIND), and 14 subjects without neurological diseases served as controls.

### Biomaterials and Flow Cytometry

Peripheral blood mononuclear cells (PBMC) were isolated by density gradient centrifugation using lymphocyte separation medium (PAA Laboratories, Pasching, Austria) as described before (Schwab et al., J. Immunol. 184(9):5368-5374 (2010)). Flow cytometry analysis of CSF was performed as described previously (Schwab et al., Mult. Scler. 18(3):335-344 (2012)). Ex vivo isolated or cultured cells were washed with phosphate-buffered saline (PBS) supplemented with 0.1% bovine serum albumin (BSA) and stained with fluorescence-labeled monoclonal antibodies (Mab) together with blocking mouse IgG (Sigma-Aldrich, Hamburg, Germany) at 4° C. for 30 min. Cells then were measured on a FACSCalibur (BD Biosciences, Heidelberg, Germany) and Gallios™ as well as Navios™ Flow Cytometer (Beckman Coulter, Krefeld, Germany) and analyzed using FlowJo (Tree Star, Ashland, Oreg., USA) and Kaluza (Beckman Coulter) software. MAb used in the study are shown in Table 1. The flow cytometry anti-CD49d mAb (clone 9F10) enables a simultaneous labeling of CD49d and bound natalizumab (secondarily labeled with IgG, data not shown). Human brain microvascular endothelial cells (HBMECs) were grown to confluency and were trypsinized. The cells were washed once in PBS containing 0.1% sodium azide and 1% bovine serum albumin (FACS buffer) and incubated for 15 min with anti-human monoclonal antibodies. After incubation the cells were again washed once with the FACS buffer and resuspended in 200 μl of FACS buffer. The anti-human monoclonal antibodies used for the HBMEC stainings are given in Table 2. The respective isotype controls (mouse IgG1 and Mouse IgG2a) were bought from BioLegend/Becton Dickinson Biosciences or Exbio. Data were acquired on Gallios™ flow cytometer (Beckman Coulter) and analyzed using Kaluza software, version 1.2 (Beckman Coulter).

TABLE 1
Monoclonal Antibodies Used for Flow Cytometry

| Antibody Against: | Clone | Manufacturer |
|---|---|---|
| CD3 | UCHT1 | Beckman Coulter |
| CD4 | 13B8.2 | Beckman Coulter |
| CD8 | B9.11 | Beckman Coulter |
| CD11a | HI111 | BD Pharmingen |
| CD14 | MoP9 | BD Biosciences |
| CD19 | HIB19 | BD Biosciences |
| CD31 | WM59 | BioLegend |
| CD45 | J33 | Beckman Coulter |
| CD45RA | HI100 | BD Biosciences |
| CD49d | 9F10 | BioLegend |
| CD54 | MEM-111 | BioLegend |
| CD56 | NCAM16.2 | BD Biosciences |
| CD62E | HAE-1F | BioLegend |
| CD62L | DREG-56 | BioLegend |
| CD62P | AK6 | Acris |
| CD106 | STA | BioLegend |
| CD146 | P1H12 | BD Biosciences |
| CD162 | KPL-1 | BioLegend |
| CD197 (CCR7) | 3D12 | BD Biosciences |

TABLE 2
Monoclonal Antibodies Used for Immunohistochemistry

| Antibody Against: | Clone | Manufacturer |
|---|---|---|
| CD8 | 144B | Abcam |
| CD54 | 3H1547 | Acris |
| CD62P | 1E3 | Santa Cruz |
| CD106 | 1.4C3 | Acris |
| CD146 | EPR3208 | Millipore |

### Immunohistochemistry

For histochemical studies 4 μm thick formalin fixed paraffin embedded (FFPE) tissues sections were stained with luxol fast blue for myelin integrity and H&E for inflammatory infiltrates. For immunohistochemical studies the antibodies used were anti-CD68, CD106 (VCAM1, mouse-IgG1, 1.4C3, Acris) and CD62P (P-Selectin, mouse IgG2a, 1E3, Santa Cruz). The secondary antibody (Envision™+Dual Link System-HRP, DAKO) was HRP conjugated and diaminobenzidine (DAKO) was used as a chromogenic substrate. Antigen retrieval was done using EnVision™ FLEX Target Retrieval Solution, Low pH (DAKO) and endogenous peroxidase activity was blocked with 3% hydrogen peroxide in methanol. In total three MS subjects and three controls were used for the staining.

### Capillary Flow Chamber

To investigate leukocyte adhesion, we used a human flow chamber system. Briefly, glass capillaries were coated with P-selectin (20 μg/ml, R&D Systems, Minneapolis, Minn.), VCAM-1 (20 μg/ml, R&D Systems) or P-selectin (20 μg/ml, R&D Systems), VCAM-1 (20 μg/ml, R&D Systems) and SDF1 (20 μg/ml, R&D Systems) for 2 h. Chambers were blocked with 1% casein (Fisher Scientific, Waltham, Mass.) for 1 h and afterwards perfused with isolated CD4 cells at a constant shear stress of 1 dynes/cm². To investigate the capturing of CD4 cells, flow chambers were coated with P-selectin (20 μg/ml) alone and were perfused with isolated CD4 cells at a constant shear stress of 1 dynes/cm² for 2 min, and, subsequently, the number of rolling cells per field of view was determined Videos were obtained with an inverted TS 1000 transmission light microscope (Nikon) equipped with a 10×/0.25 objective and a digital camera (Pixelfly, Cooke Corporation, Romulus, Mich.).

### Parallel Plate Flow Chamber

Primary human brain microvascular endothelial cells (HBMEC) were purchased from ScienCell Research Laboratories (San Diego, Calif., USA) and grown to confluence on 35 mm culture dishes. Cells were stimulated with TNFα (10 nM) for 16 hours. Human CD4⁺ T-cells were counted and resuspended in perfusion buffer (Medium 199 supplemented with FCS and HEPES). Cells were perfused through the parallel plate flow chamber (Glykotech, Gaithersburg, Md., USA) at a constant shear stress of 0.25 dyne/cm² for 8 minutes. Cells were observed using an inverted microscope (Nikon TS-1000) equipped with a 10×/0.25NA objective and a digital camera. Videos were recorded using CamWare software (PCO Ag, Kelheim, Germany) and rolling and adherent cells per field of view were analyzed.

### Statistical Analysis

Statistical significance of differences between two groups was determined using unpaired Student's t-test except for comparisons between peripheral blood and CSF of the same subject, where the paired Student's t-test was used. Differences were considered statistically significant with p* values <0.05, with p**<0.01 and p***<0.001. Software for statistical and correlation assessment was Prism 5 (GraphPad, La Jolla, Calif., USA).

### Example 1

### Cerebrospinal Fluid Isolated from MS Subjects Under Long-Term Treatment with Natalizumab Reflects a Normalization of the Central Immune Response

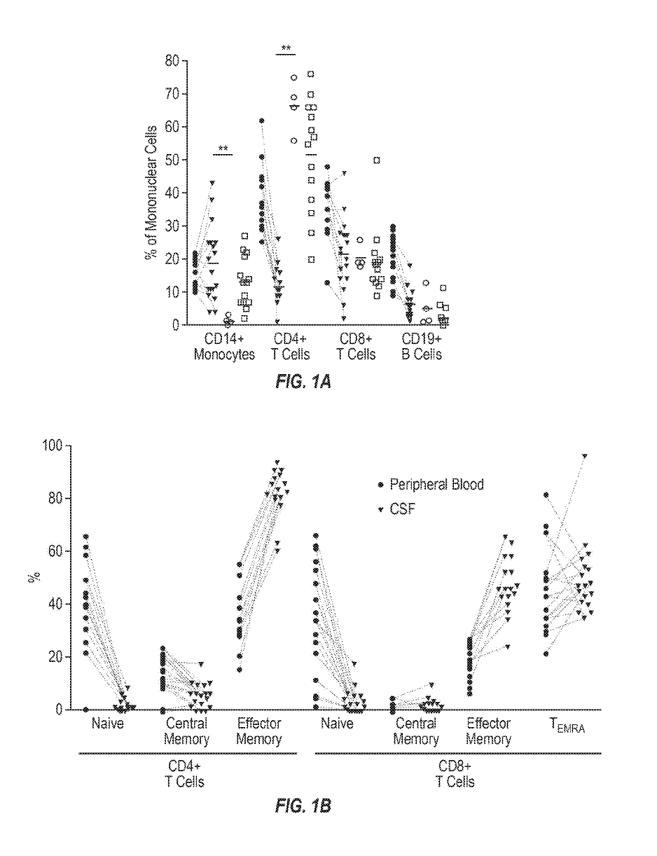

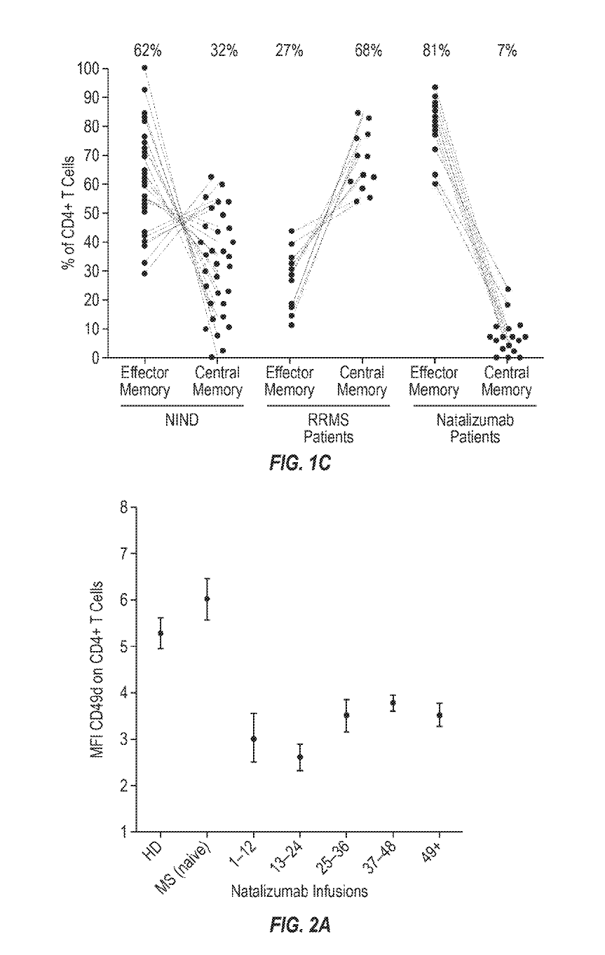

FIG. 1 (A-C) show changes in the cerebrospinal fluid under long-term treatment with natalizumab reflect a normalization of the central immune response in MS subjects. FIG. 1A shows the CSF composition of long-term treated natalizumab subjects (filled triangles; n=18) when compared with corresponding peripheral blood samples (filled circles) and CSF values from either untreated RRMS subjects (clear circles; n=4) or control subjects (clear squares; n=14). FIG. 1B shows the effector/memory compartments of CD4+ and CD8+ T cells in the CSF of long-term treated natalizumab subjects (n=18). Naïve (CD45RA+CCR7+), Central memory (CD45RA-CCR7+), and effector memory (CD45RA-CCR7−) CD4+ and CD8+ T cells are shown. Additionally, in the case of CD8+ T cells, TEMRA (CD45RA+CCR7−) are shown. FIG. 1C shows the CSF composition concerning effector memory CD4+ T cells (EM) and central memory CD4+ T cells (CM) in non-inflammatory neurological diseases (NIND; n=33), RRMS subjects (n=12), and MS subjects under long-term natalizumab therapy (n=18).

Flow cytometric analysis of PBMC from long-term natalizumab-treated (LTNT, ≧18 months of continuous treatment) RRMS subjects revealed that the relative level of all major immune cell subsets was within normal limits. However, assessment of immune cells derived from cerebrospinal fluid of clinically stable LTNT subjects revealed clear differences between subsets compared to treatment-naïve, stable RRMS subjects: the percentage of CD14⁺ monocytes was elevated in natalizumab-treated subjects (18.9% vs 1.4%), and, was similar to that of control subjects without any neurological disease (13.2%). The percentage of CD4⁺ T cells was reduced (11.8% vs 66.5%) while CD8⁺ T cells were unchanged, resulting in a reversed CD4:CD8 ratio when compared to untreated MS subjects (0.54 vs 3.24) (FIG. 1A). Looking at the CD4⁺ and CD8⁺ T-cell effector-memory compartments, there was an expected shift towards memory cells in the CSF compared to the peripheral effector-memory compartments (FIG. 1B) Importantly, also here the ratio between CD4 effector-memory (EM) and central-memory (CM) cells in LTNT subjects (EM:CM 81:7) was similar to non-inflammatory neurological diseases (62:32) and inverted when compared to treatment-naïve, stable RRMS subjects (27:68) (FIG. 1C). These results suggest that effector memory cells have mechanisms that allow better access to the CNS than central memory cells in the presence of natalizumab.

### Example 2

### CD49d Expression in Peripheral and Central T-Cell Compartments Under Long-Term Natalizumab Therapy

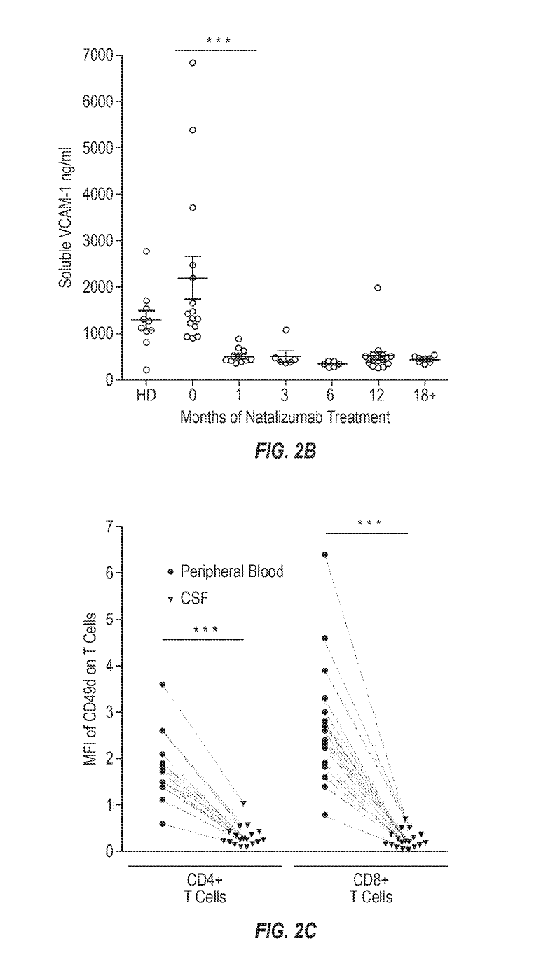

FIG. 2 (A-C) shows CD49d expression in peripheral and central T-cell compartments under long-term natalizumab therapy. FIG. 2A shows the expression of CD49d on peripheral CD4+ and T cells of either healthy controls (n=26) or MS subjects before the start of natalizumab therapy (MS naïve; n=33), or during natalizumab therapy (n=486). FIG. 2B shows soluble VCAM-1 in the serum of healthy controls (n=10), MS subjects before treatment (n=15) and during treatment with natalizumab (n=49). FIG. 2C shows a comparison of the expression of CD49d on peripheral and CSF CD4+ and CD8+ T cells of long-term treated natalizumab subjects (n=18).

Next to changes in cell populations, we assessed the expression of the molecule targeted by natalizumab, CD49d, and its ligand VCAM-1. Natalizumab treatment induced a rapid decrease of CD49d surface expression on peripheral blood CD4⁺ T cells, which was not accompanied by a downregulation in gene expression (FIG. 2A). Also, natalizumab treatment substantially reduced the soluble form of the CD49d ligand, VCAM-1, in serum with the first infusion (FIG. 2B). Surprisingly, CD49d expression was not detectable on CSF T cells of LTNT subjects, even when compared to the downregulated/low CD49d level on peripheral T cells (FIG. 2C).

### Example 3

### Natalizumab Treatment Induces Upregulation of PSGL-1

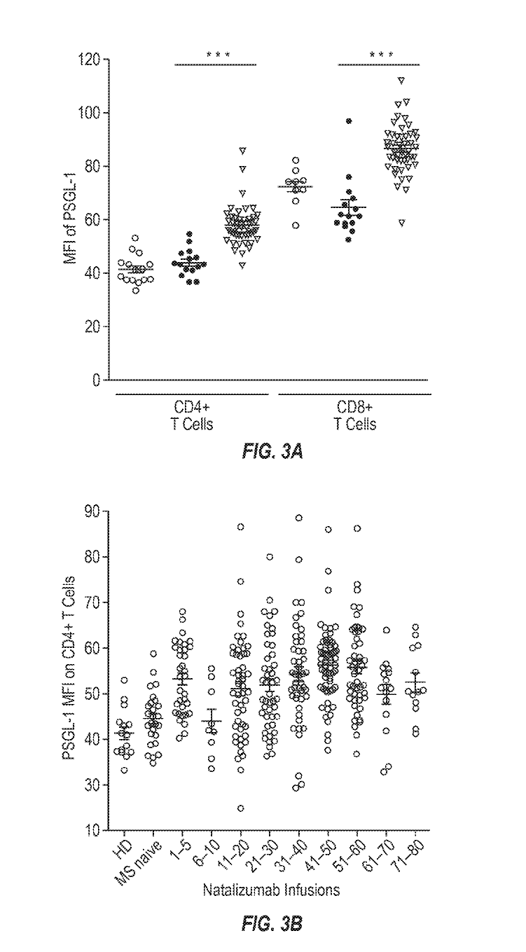

FIGS. 3 (A & B) shows natalizumab treatment induces upregulation of PSGL-1. FIG. 3A shows surface expression level of PSGL-1 on either healthy controls (white; n=15), subjects before (black; n=15) or after long-term natalizumab therapy (gray; n=47) on CD4+ and CD8+ T cells. FIG. 3B shows time courses of PSGL-1 expression before (MS naïve; n=26) and during natalizumab treatment (n=330) are shown for CD4+ T cells when compared with healthy controls (n=15).

The adhesion molecule P-selectin glycoprotein ligand-1 (PSGL-1) was strongly upregulated on CD4⁺ and CD8⁺ T cells after LTNT (CD4MFI 58.12±7.35; CD8MFI 86.79±9.23) when compared to subjects before treatment (CD4MFI 43.97±5.10; CD8MFI 64.53±10.85) (FIG. 3A). There was a clear and reproducible time kinetic to the PSGL-1 upregulation, peaking after four years of treatment (FIG. 3B).

### Example 4

### Molecular Localization of CD49d and PSGL-1 on CD4⁺ T Cells

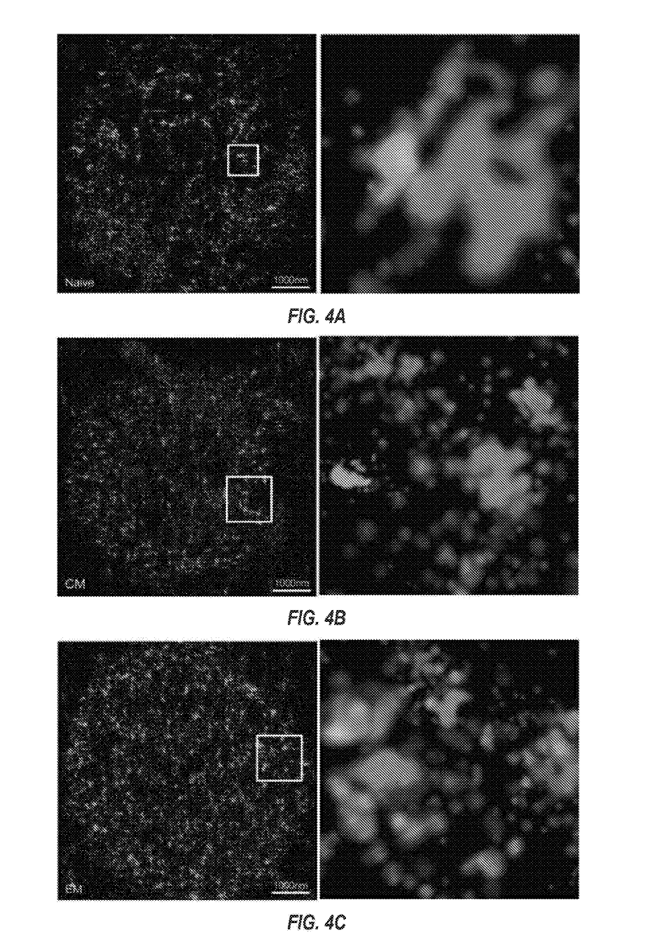

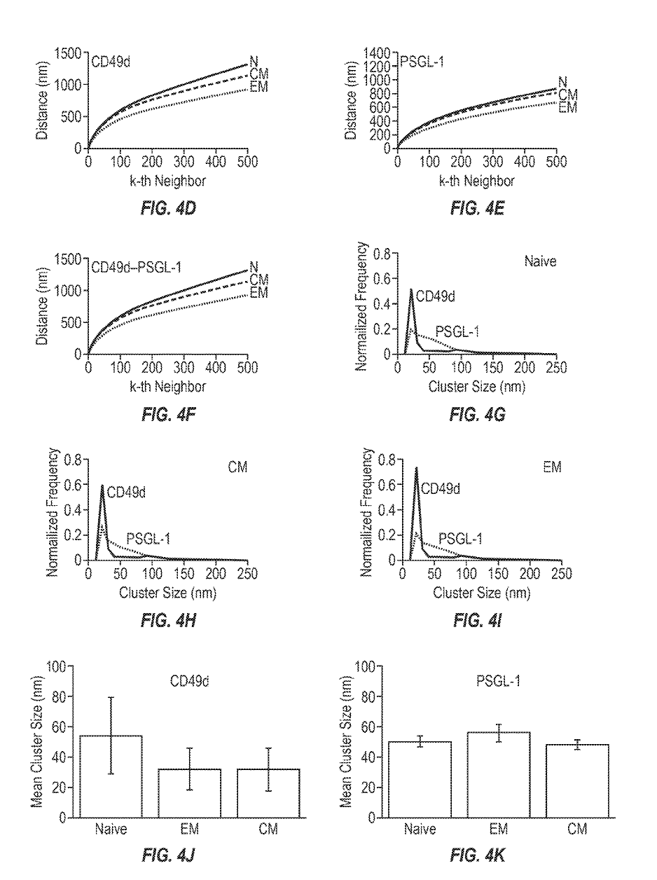

FIG. 4 (A-K) shows the molecular distribution of CD49d and PSGL-1 on CD4+ T cells. Shown is one representative naïve (FIG. 4A), central-memory (FIG. 4B), and effector-memory (FIG. 4C) CD4+ T cell with CD49d labeled in green and PSGL-1 labeled in red (co-localization in yellow). Each 3D insert shows an exemplary cluster of molecules. Shown are nearest neighbor analyses of CD49d (FIG. 4D), PSGL-1 (FIG. 4E) and their combination (FIG. 4F), the cluster size of CD49d (black) and PSGL-1 (red) on naïve (FIG. 4G), CM (FIG. 4H), and EM (FIG. 4I). FIG. 4J shows the mean average cluster size of CD49d on naïve (1), EM (2), and CM (3), whereas FIG. 4K shows the same for PSGL-1 cluster size.

After we saw the highly significant changes induced by natalizumab in the expression of CD49d and PSGL-1 on CD4⁺ T cells, we investigated whether we could exclude the possibility that the binding of natalizumab to CD49d could influence other molecules, such as PSGL-1, that may be co-localized. We used direct stochastical optical reconstruction microscopy (dSTORM) on single cells to measure the distribution of these two molecules on naïve, central-, and effector-memory CD4⁺ T cells (FIG. 4 A-C). There was no co-localization between CD49d and PSGL-1 on naïve, only little co-localization on CM and strong co-localization only on EM cells. The nearest neighbor analysis showed smaller distances between molecules of CD49d (FIG. 4D) as well as PSGL-1 (FIG. 4E) molecules on memory CD4⁺ T cells compared with naïve CD4⁺ T cells with an especially strong expression on EM cells, fitting to flow cytometry stainings indicating higher expression of these molecules. Therefore, the molecules CD49d and PSGL-1 were in closer proximity to each other, especially on EM cells (FIG. 4F). Concerning the appearance of molecular clusters, CD49d only showed rare clusters on naïve cells and no clusters on memory cells, whereas PSGL-1 strongly clustered on all three subpopulations (FIG. 4G-K). As a consequence, the average cluster size of PSGL-1 was higher than the cluster size of CD49d (FIG. 4J-K). The lack of co-localization of CD49d and PSGL-1 on naïve and CM cells suggests that the change in PSGL-1 expression is not likely to be an artifact due to natalizumab binding.

### Example 5

### Expression of VCAM-1 and P-Selectin on Possible Routes of Entry in Multiple Sclerosis Subject and Control Tissues

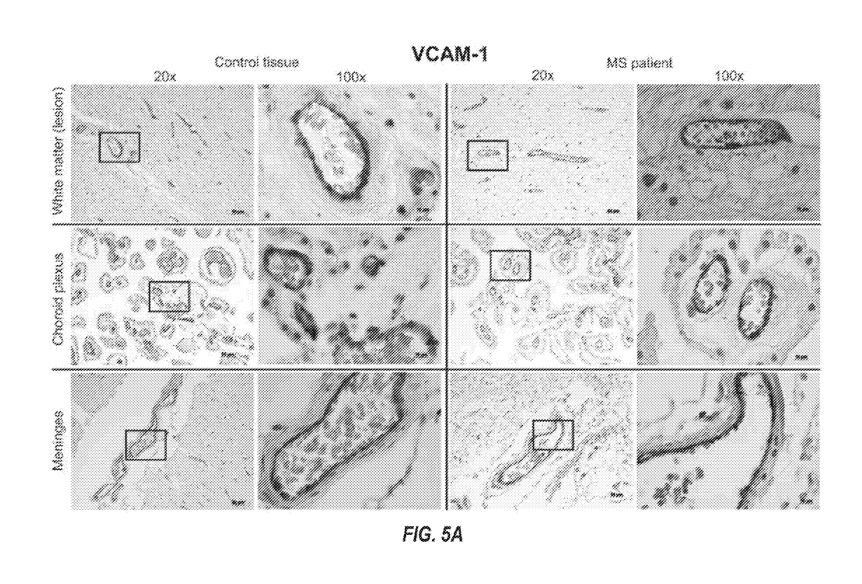

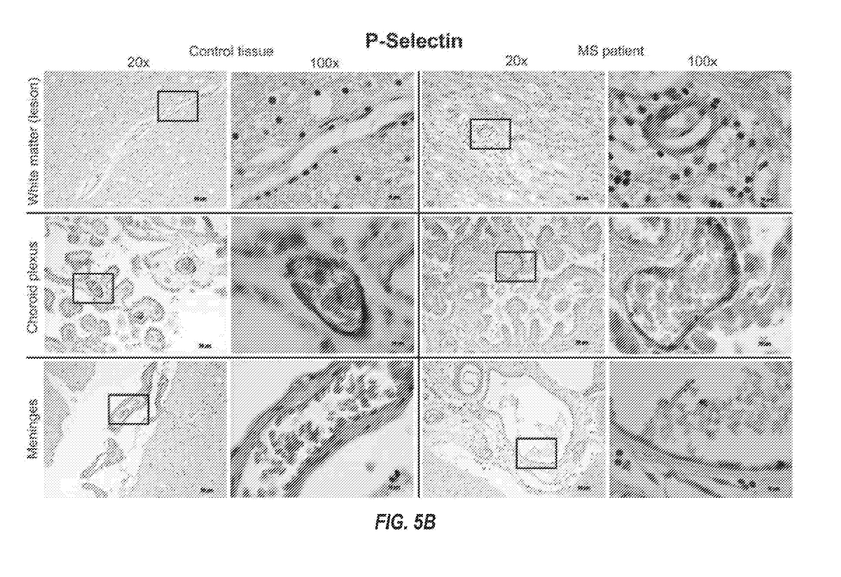

FIGS. 5 (A & B) shows expression of VCAM-1 and P-selectin on possible routes of entry in multiple sclerosis subject and control tissues. VCAM-1 and P-selectin expression in the parenchymal (lesion area), choroid plexus and meningeal blood vessels are shown in FIGS. 5A and B, respectively. The insert shows 100× magnification. DAB was used as chromogenic substrate and hematoxylin for counter stain. Table 3 provides an immuno-histological assessment of the expression levels of P-Selectin and VCAM-1.

To assess the relevance of our findings for the migration of T cells over different blood-CNS barriers in vivo, we stained for the primary ligands of CD49d and PSGL-1 (i.e., VCAM-1 and P-Selectin) in the tissue of MS subjects and controls. The regions of interest were the meninges to assess the blood-leptomeningeal barrier, (normal-appearing/lesional) white matter to assess the direct migration from blood vessels to CNS tissue/traditional blood-brain barrier, and the choroid plexus to assess the blood-CSF barrier. We showed that there are varying degrees of VCAM-1 on all the endothelial barriers, but the expression of P-selectin was much more restricted. There was no expression of P-selectin on white matter (lesion) vessels, a very low expression on meningeal vessel endothelium and pronounced expression on choroid plexus endothelium. Of note, the expression on the choroid plexus endothelium in the tissue of MS subjects was generally more pronounced than in control tissues (FIG. 5).

TABLE 3
Immuno-histological assessment of the expression
levels of P-Selectin and VCAM-1

| | Controls | | | MS subjects | | |
|---|---|---|---|---|---|---|
| | Choroid | | White | Choroid | | White |
| | plexus | Meninges | matter | plexus | Meninges | matter |
| P-selectin | −/++ | + | − | +/++ | + | − |
| VCAM-1 | +++ | ++ | ++ | +++ | ++ | + |

### Example 6

### Influence of Natalizumab on Rolling and Adherence of CD4⁺ T Cells to the Ligands of CD49d and PSGL-1 (i.e., VCAM-1/P-Selectin)

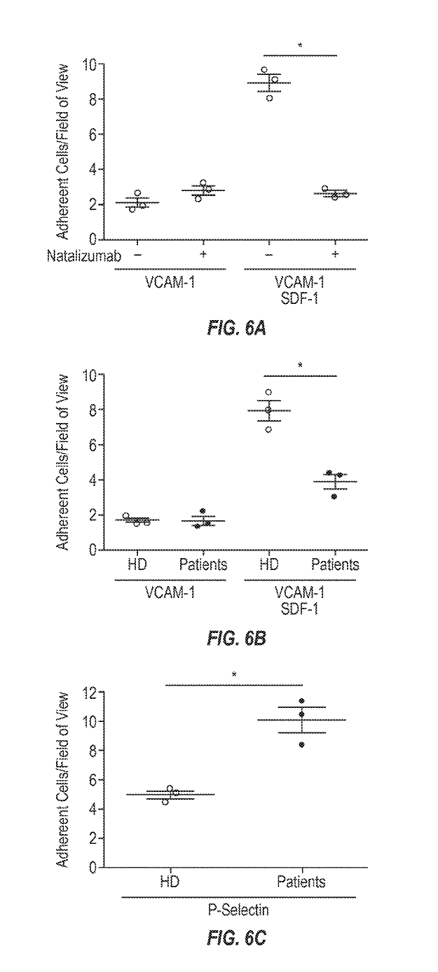

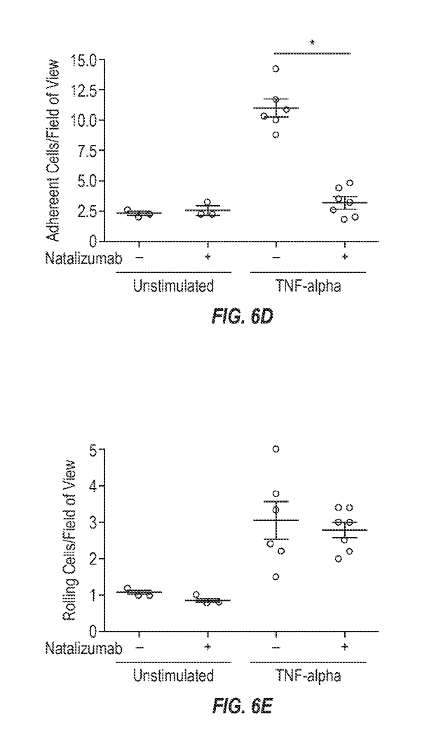

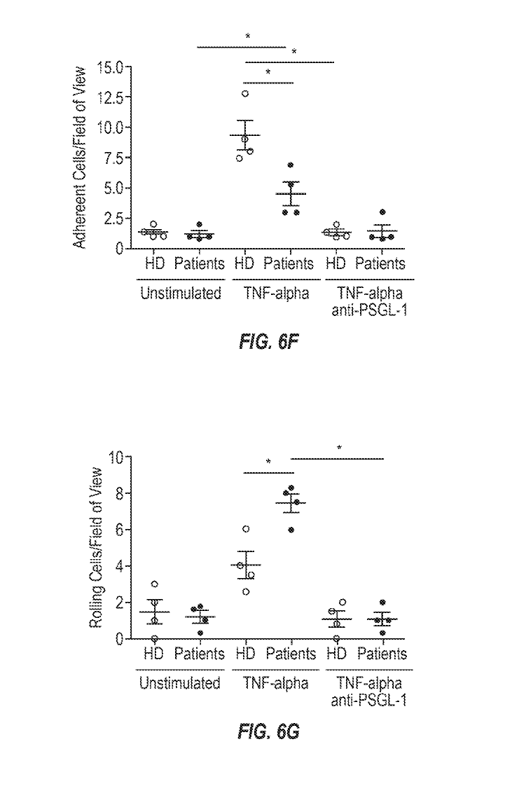

FIG. 6 (A-G) show the influence of natalizumab treatment on rolling and adherence of CD4+ T cells to the ligands of CD49d and PSGL-1 (i.e., VCAM-1/P-selectin).

FIG. 6A shows adherent CD4+ T cells/field of view of a VCAM-1 coated capillary flow chamber with and without SDF-1 and with and without natalizumab (n=4). FIG. 6 shows adherent CD4+ T cells of natalizumab-treated subjects and controls/field of view on a VCAM-1 coated capillary flow chamber with and without SDF-1 (n=4). FIG. 6C shows captured CD4+ T cells of healthy controls and natalizumab-treated subjects/field of view on P-selectin coated capillary flow chambers (n=4). Adherent or rolling CD4+ T cells/field of view on parallel plate flow chambers coated with either non-inflamed or TNF-alpha inflamed primary endothelial cells with and without addition of natalizumab (n=4) are shown in FIGS. 6 D and E, respectively. Adherent rolling CD4+ T cells of natalizumab-treated subjects and controls/field of view on parallel plate flow chambers coated with either non-inflamed or TNF-alpha inflamed primary endothelial cells with and without PSGL-1 blocking antibodies (n=4) are shown in FIGS. 6 F and G, respectively.

Both CD49d and PSGL-1 are molecules critically involved in the first steps of lymphocyte extravasation: tethering, rolling, and adhesion (reviewed in Engelhardt and Ransohoff, Nat. Rev. Immunol. 12(9):623-635 (2012); Engelhardt and Ransohoff, Trends Immunol. 33(12):579-589 (2012)). We thus assessed whether the natalizumab-mediated blockade of CD49d and the subsequent upregulation of PSGL-1 are functionally relevant under shear-flow conditions. In vitro treatment of healthy controls' CD4⁺ T cells with natalizumab abrogated the interaction of CD49d with VCAM-1 in a glass capillary-based system and in line with the established view, adhesion to VCAM-1 required the conformational change of VLA-4 by a chemokine stimulus (SDF-1/CXCL12) (FIG. 6A). We subsequently showed that this reduced adhesion was also evident in CD4⁺ T cells freshly isolated from the blood of subjects under LTNT (FIG. 6B), demonstrating the treatment efficacy in vitro. Analysis of P-selectin (PSGL-1 receptor) coated glass capillaries revealed that the capturing capacity of subject cells was significantly elevated compared with healthy control cells (FIG. 6C). Primary human brain-derived microvascular endothelial cells (HBMEC) grown to confluency were used in the next step to mimic the natural expression of the CD49d- and PSGL-1 binding partners. We showed that CD4⁺ T cells of healthy controls cannot adhere to the endothelium when pre-incubated with natalizumab, proving the relevance of the VLA-4/VCAM-1 interaction in our system (FIG. 6D). Blockade of CD49d had no influence on the rolling capacity of CD4⁺ T cells over endothelium (FIG. 6E). Firm adherence to the endothelial layer was reduced, but not completely abrogated in natalizumab-treated subjects and both subject—as well as control cells showed no residual adherence in the presence of a PSGL-1 blocking antibody (FIG. 6F). As a final step, we showed that the rolling of CD4⁺ T cells over endothelium, which was shown in the capillary system (FIG. 6E) to be independent of CD49d, was more pronounced in natalizumab-treated subjects and this rolling could again be prevented by addition of a PSGL-1 blocking antibody (FIG. 6G).

### Example 7

### T_{H}17 Cells can Use MCAM to Adhere to Endothelium Independently of VCAM-1/VLA-4 Interactions

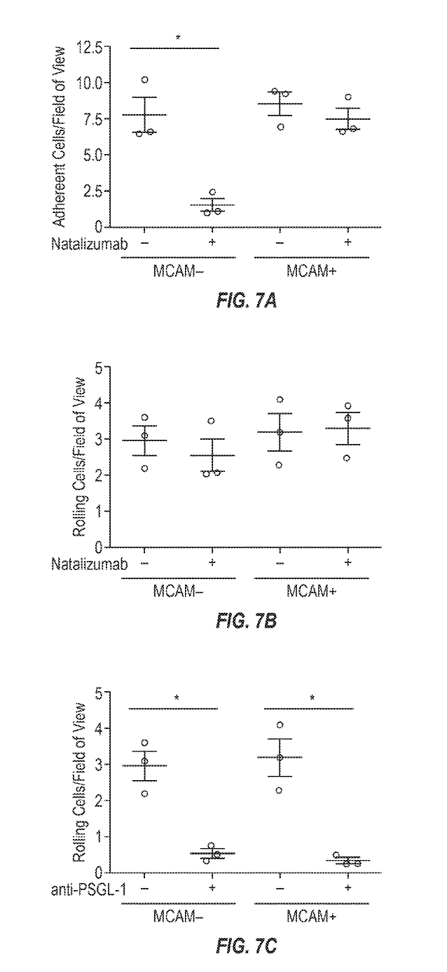

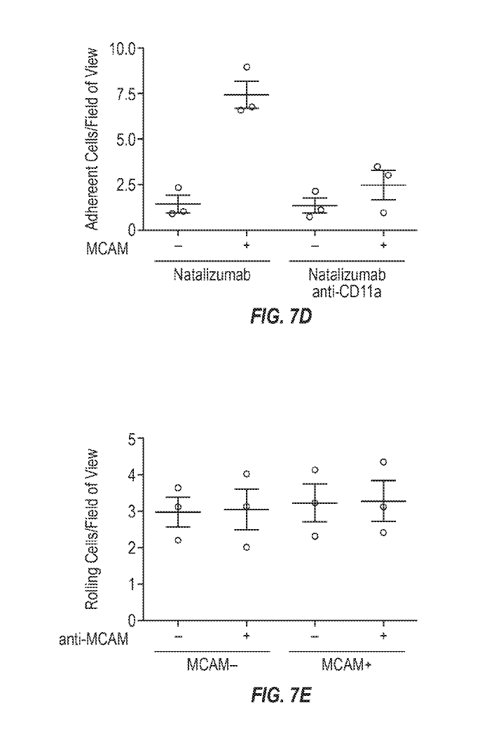

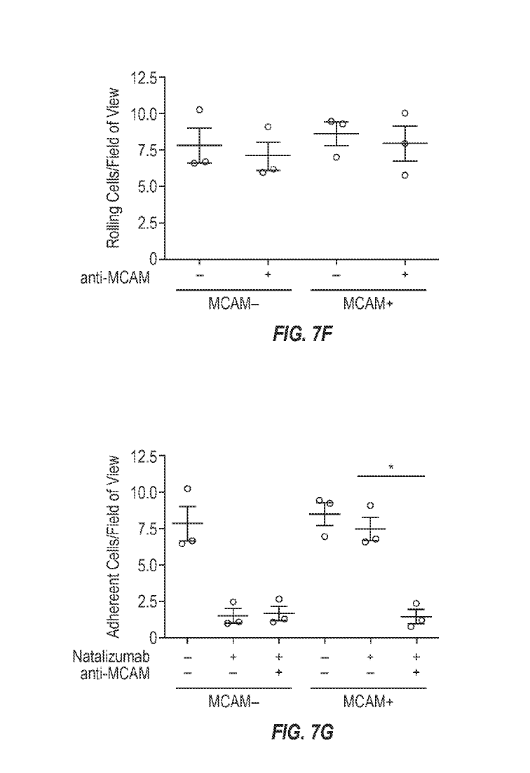

FIG. 7 (A-G) shows T_{H}17 cells can use MCAM for firm adhesion to endothelium. Shown are CD4+CD45RO+MCAM+/− T cells on parallel plate flow chambers coated with TNF-alpha inflamed primary endothelial cells: adherent cells/field of view with and without addition of natalizumab (n=3) (FIG. 7A); rolling cells/field of view with and without addition of natalizumab (n=3) (FIG. 7B); rolling cells/field of view with and without blockade of PSGL-1 (n=3) (FIG. 7C); adherent cells/field of view with and without addition of natalizumab and blockade of CD11a (n=3) (FIG. 7D); rolling cells/field of view with and without blockade of MCAM (n=3) (FIG. 7E); adherent cells/field of view with and without blockade of MCAM (n=3) (FIG. 7F) and, adherent cells/field of view with and without addition of natalizumab and blockade of MCAM (n=3) (FIG. 7G).

As there was still residual adherence to endothelium in LTNT subjects (FIG. 6D), the CSF cells in those subjects did not express CD49d (FIG. 2C), recent studies have shown that in contrast to T_{H}1 cells, T_{H}17-polarized cells can induce EAE in CD49d deficient mice (Rothhammer et al., J. Exp. Med. 208(12):2465-2476 (2011)), and that melanoma cell adhesion molecule (=MCAM, CD146) is a specific marker for T_{H}17 cells in humans (Larochelle et al., Brain 135(Pt 10):2906-2824 (2012); Flanagan et al., PLoS One 7(7):e40443 (2012)), we subsequently focused on T_{H}17/MCAM⁺ cells. We used our in vitro assay to challenge the hypothesis that T_{H}17 cells can still enter the CSF under LTNT in the human system and could show that blockade of CD49d by natalizumab abrogated the adherence of MCAM⁻ memory cells (CD3⁺CD4⁺CD45RO⁺MCAM⁻), but did not interfere with the adhesion of MCAM⁺ memory cells (FIG. 7A). We also tested this hypothesis additionally ex vivo in two proof-of-concept subjects, where we had access to CSF material and showed that the percentage of MCAM⁺ cells of CD3⁺CD4⁺CD45RA⁻ cells was much higher in the LTNT subject when compared to the stable MS subject without natalizumab treatment (19.8 vs. 3.2%), suggesting a prevalence of T_{H}17 cells in the CSF of LTNT subjects. These T_{H}17 cells, as shown above for whole CD4⁺ T cells in FIG. 2C, also did not express CD49d in LTNT subjects. There was no enhanced in vitro rolling of MCAM⁺ cells and again blockade of CD49d did not influence capturing (FIG. 7B). PSGL-1 blockade abrogated rolling in both populations (FIG. 7C). By blocking CD11a in addition to CD49d, we could reduce adhesion of MCAM⁺ cells (FIG. 7D). Finally, we blocked their signature molecule (MCAM) in vitro with an antibody developed for a potential clinical application (i.e., blocking the function of T_{H}17 cells in vivo to interfere with autoimmune processes). MCAM blockade alone did not influence rolling (FIG. 7E) or adherence (FIG. 7F) to endothelial cells. However, the blockade of CD49d and MCAM together abrogated the adherence of MCAM⁺ cells to endothelium (FIG. 7G).

### Example 8

### In Situ Detection of MCAM⁺ T Cells in MS Lesions Suggesting a Role of T_{H}17 Cells in the Pathogenesis of MS

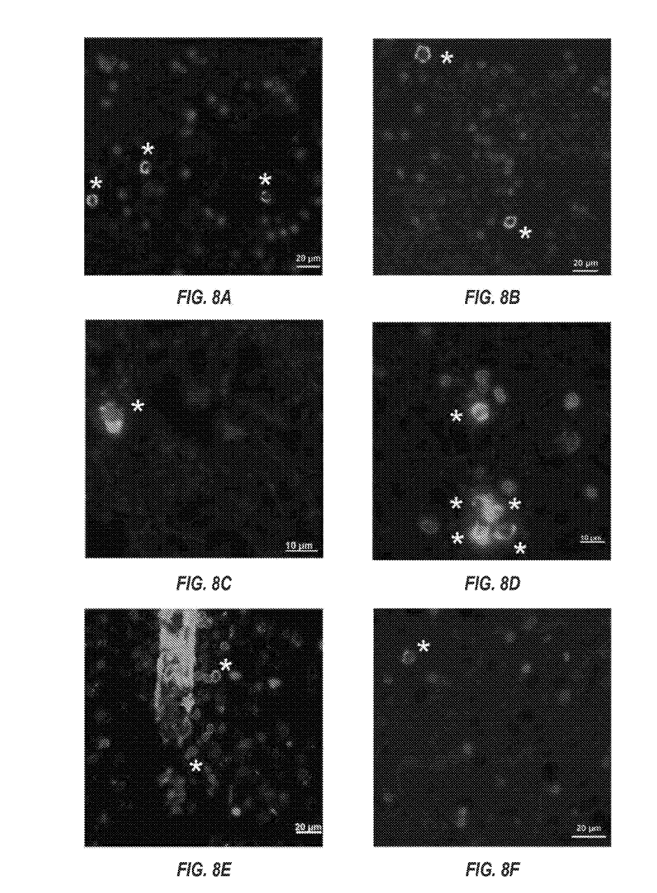

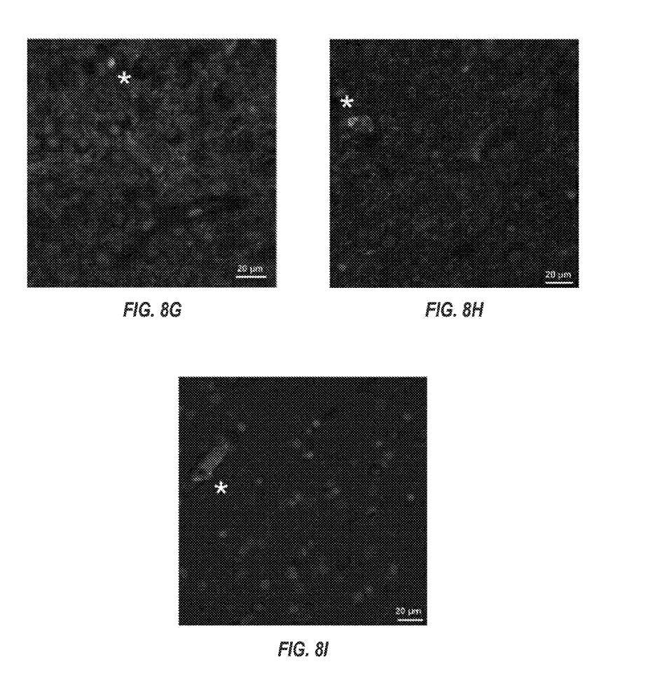

FIG. 8 (A-I) shows MCAM+ lymphocytes in active MS lesions. FIGS. 8A-E show examples of active white matter lesions from three MS subjects autopsies with MCAM shown in green and DAPI shown in blue. To exclude the possibility of MCAM+CD8+ lymphocytes, FIG. 8E shows a double staining for MCAM (green) and CD8 (red) with DAPI counter staining, showing the prominent MCAM expression of endothelial cells and two MCAM+CD8− lymphocytes near the blood vessel. FIGS. 8F-I show examples of MCAM+ cells in gray matter from three MS subjects' autopsies. MCAM+ lymphocytes are marked with asterisks.

To support our observations with T_{H}17 in MS pathogenesis, MS white matter lesions were assessed for the presence of MCAM⁺ lymphocytes (FIG. 8A-D). The MCAM⁺ cells were shown to be Iba1⁻, CD68⁻, GFAP⁻, NeuN⁻, and CD8⁻ (FIG. 8E). MCAM⁺ cells could also be detected in gray matter of MS subjects (FIG. 8F-I) with several cells in direct apposition to blood vessel walls (FIG. 8H-I).

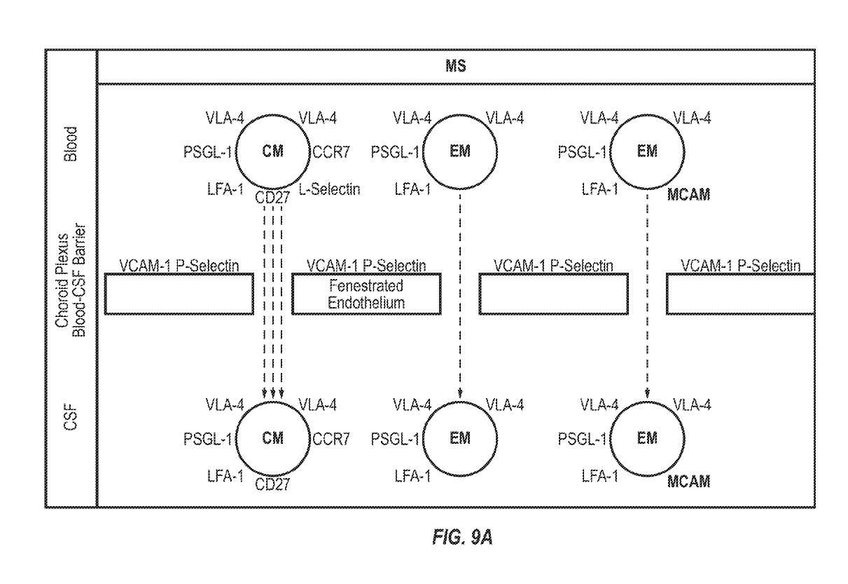

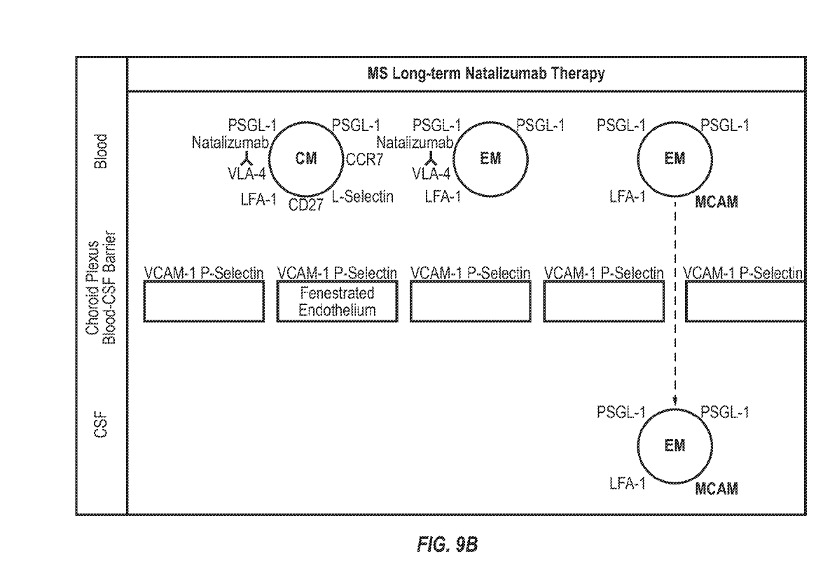

The blockade of both VLA-4 and MCAM succeeded in reducing the adhesion of MCAM⁺ cells, providing evidence T_{H}17 cells can use both VLA-4 or their signature molecule MCAM to mediate firm adhesion to endothelium, introducing MCAM as part of the migratory cascade into target tissue, specifically for T_{H}17 cells. MCAM binds to a form of laminin produced specifically by endothelial cells (laminin α4), and is part of the endothelial matrix. However, laminin α4 may also be presented on the luminal surface of endothelial cells and contribute to early events within the adhesive cascade and subsequently facilitate migration across the vessel wall. The fact that there is no residual adherence in our system in the presence of an antibody against MCAM that blocks laminin binding is consistent with this hypothesis. Our results show that T_{H}17 cells have additional adhesive mechanisms that may facilitate CNS entry even in the presence of natalizumab. LFA-1 likely plays an important role, consistent with the findings of Rothhammer et al., J. Exp. Med. 208(12):2465-2476 (2011) in EAE using conditional α4 integrin knock-out mice, and MCAM/laminin as well consistent with results from Flanagan et al., PLoS One 7(7):e40443 (2012) and Larochelle et al., Brain 1353(Pt 10):2906-2924 (2012). These results provide evidence that most cells still being able to enter the CSF in natalizumab-treated subjects are highly likely T_{H}17 cells, either suggesting a role of this population not only in pathology, but also in CNS immune surveillance or simply suggesting that the presence of T_{H}17 cells alone without the presence of T_{H}1 cells does not mediate pathology in MS, unlike in EAE where T_{H}17 cells have been shown to induce EAE symptoms (Rothhammer et al., J. Exp. Med. 208(12):2465-2476 (2011)). This result provides an explanation of why some subjects exhibit strong relapses after cessation of treatment with natalizumab (Melis et al., Neurol. Sci. 35(3):401-408 (2014)), in that T_{H}1 cells can again gain entry into the CNS and the T_{H}17 cells, which are already in situ, exacerbate tissue damage and clinical symptoms. This data set provides evidence that a blockade of VLA-4 and MCAM together can be an even stronger treatment than VLA-4 blockade alone or could be an efficient treatment alternative in cases, where the blockade of T_{H}1 migration is insufficient to alleviate clinical symptoms (e.g., cases with a strong T_{H}17-biased presentation such as neuromyelitis optica (Varrin-Doyer et al., Ann. Neurol. 72(1):53-64 (2012)). The findings of our study are summarized in a schematic overview (FIG. 9). Concerning progression and gray matter atrophy in MS, our data provide evidence that T_{H}17 cells can mediate residual progression under natalizumab, because we also find these cells in gray matter and their entry is not blocked by the treatment. This result provides evidence that progression in SPMS and PPMS also has a T_{H}17 component and that a combined blockade of VLA-4 and MCAM can be a valid treatment option for these diseases.

What is claimed is:

1. A method of treating or effecting prophylaxis of an autoimmune disease, comprising administering an MCAM antagonist to a subject having or at risk of multiple sclerosis who also receives an alpha-4 integrin antagonist, wherein the MCAM antagonist and alpha-4 integrin antagonist are provided in a regime effective for treatment or prophylaxis of the autoimmune disease.

2. A method of treating or effecting prophylaxis of an autoimmune disease, comprising administering an alpha-4 integrin antagonist to a subject having or at risk of multiple sclerosis who also receives an MCAM-antagonist, wherein the alpha-4 integrin antagonist and the MCAM antagonist are provided in a regime effective for treatment or prophylaxis of the autoimmune disease.

3. The method of claim 1 or claim 2, wherein the autoimmune disease is multiple sclerosis.

4. The method of any one of claims 1-3, wherein the alpha-4 integrin antagonist antagonizes alpha-4 integrin binding to VCAM-1.

5. The method of claim 4, wherein the alpha-4 integrin antagonist specifically binds to alpha-4 integrin.

6. The method of claim 5, wherein the alpha-4 integrin antagonist is a monoclonal antibody.

7. The method of claim 6, wherein the monoclonal antibody is natalizumab.

8. The method of any one of claims 1-7, wherein the MCAM-antagonist antagonizes MCAM binding to laminin-alpha-4.

9. The method of claim 8, wherein the MCAM antagonist is a monoclonal antibody that specifically binds to MCAM.

10. The method of claim 9, wherein the MCAM antagonist is a monoclonal antibody that specifically binds to laminin-alpha-4.

11. The method of claim 10, wherein the MCAM antibody is 1749 or 2120, or a chimeric, veneered, or humanized version thereof.

12. The method of any one of claims 1-11, wherein the alpha-4 integrin antagonist is natalizumab and the MCAM antagonist is 1749 or 2120, or a chimeric, veneered, or humanized version thereof.

13. The method of any one of claim 1-12, 28-29, or 32-34 wherein the alpha-4 integrin antagonist and MCAM antagonist are provided concurrently such that both at detectable in serum of the subject at the same time.

14. The method of claim 13, wherein the alpha-4 integrin antagonist and MCAM antagonist are provided by simultaneous infusion.

15. The method of any of claim 1-12, 28-29, or 32-34 wherein the alpha-4 integrin antagonist and MCAM antagonist are provided sequentially.

16. The method of claim 15, wherein the alpha-4 integrin antagonist is provided first, the subject developments resistance to the alpha-4 integrin antagonist and the MCAM antagonist is then provided.

17. The method of claim 15, wherein the MCAM antagonist is provided first, the subject develops resistance to the MCAM antagonist, and the alpha-4 integrin antagonist is then provided.

18. The method of claim 15, wherein a course of treatment with the alpha-4 integrin antagonist is administered first and the subject has or is at risk of relapsing remitting multiple sclerosis on initiating the course of treatment and the subject has progressed to secondary progressive multiple sclerosis on initiating a course of treatment with the MCAM antagonist.

19. The method of any one of claim 1-18, 28-29, or 32-34 wherein the alpha-4 integrin and MCAM antagonists are each provided at intervals of weekly to quarterly.

20. The method of claim 19, wherein the alpha-4 integrin and MCAM antagonists are each provided at four-weekly intervals.

21. The method of any one of claim 1-20, 28-29, or 32-34 wherein the dose of each antibody is 50-500 mg/subject.

22. The method of claim 21, wherein the dose of each antibody is 100-200 mg/subject.

23. The method of claim 21, wherein the dose of each antibody is 50-150 mg/subject.

24. A method of treating or effecting prophylaxis of an autoimmune disease involving T-cell infiltration, comprising administering to a subject having or at risk of the inflammatory disease an alpha-4 antagonist and an MCAM antagonist, wherein the alpha-4 integrin antagonist and the MCAM antagonist are provided in a regime effective for treatment or prophylaxis of the autoimmune disease.

25. The method of claim 24, wherein the autoimmune disease is multiple sclerosis, rheumatoid arthritis, Crohn's disease, inflammatory bowel disease, sarcoidosis, or psoriatic arthritis.

26. The method of claim 24 or claim 25, wherein the MCAM antagonist and alpha-4 integrin antagonist are administered sequentially with the alpha-4 integrin antagonist administered first.

27. The method of claim 24 or claim 25, wherein the MCAM antagonist and alpha-4 antagonist are administered concurrently.

28. The method of claim 12, wherein the MCAM antibody comprises a mature heavy chain variable region having the amino acid sequence of SEQ ID NO:100, a mature light chain variable region having the amino acid sequence of SEQ ID NO:86, a heavy chain constant region having the amino acid sequence of SEQ ID NO:104, and a light chain constant region having the amino acid sequence of SEQ ID NO:101.

29. The method of claim 12, wherein the MCAM antibody comprises a mature heavy chain variable region having the amino acid sequence of SEQ ID NO:93, a mature light chain variable region having the amino acid sequence of SEQ ID NO:94, a heavy chain constant region having the amino acid sequence of SEQ ID NO:104, and a light chain constant region having the amino acid sequence of SEQ ID NO:101.

30. The method of any one of claims 24-27, wherein the MCAM antagonist is a monoclonal antibody comprising a mature heavy chain variable region having the amino acid sequence of SEQ ID NO:100, a mature light chain variable region having the amino acid sequence of SEQ ID NO:86, a heavy chain constant region having the amino acid sequence of SEQ ID NO:104, and a light chain constant region having the amino acid sequence of SEQ ID NO:101.

31. The method of any one of claims 24-27, wherein the MCAM antagonist is a monoclonal antibody comprising a mature heavy chain variable region having the amino acid sequence of SEQ ID NO:93, a mature light chain variable region having the amino acid sequence of SEQ ID NO:94, a heavy chain constant region having the amino acid sequence of SEQ ID NO:104, and a light chain constant region having the amino acid sequence of SEQ ID NO:101.

32. The method of claim 12, wherein the MCAM antibody comprises a mature heavy chain variable region having the amino acid sequence of SEQ ID NO:100, a mature light chain variable region having the amino acid sequence of SEQ ID NO:86, a heavy chain constant region having the amino acid sequence of SEQ ID NO:105, and a light chain constant region having the amino acid sequence of SEQ ID NO:101.

33. The method of claim 12, wherein the MCAM antibody comprises a mature heavy chain variable region having the amino acid sequence of SEQ ID NO:93, a mature light chain variable region having the amino acid sequence of SEQ ID NO:94, a heavy chain constant region having the amino acid sequence of SEQ ID NO:104, and a light chain constant region having the amino acid sequence of SEQ ID NO:102.

34. The method of claim 12, wherein the MCAM antibody comprises a mature heavy chain variable region having the amino acid sequence of SEQ ID NO:93, a mature light chain variable region having the amino acid sequence of SEQ ID NO:94, a heavy chain constant region having the amino acid sequence of SEQ ID NO:105, and a light chain constant region having the amino acid sequence of SEQ ID NO:102.

35. The method of any one of claims 24-27, wherein the MCAM antagonist is a monoclonal antibody comprising a mature heavy chain variable region having the amino acid sequence of SEQ ID NO:100, a mature light chain variable region having the amino acid sequence of SEQ ID NO:86, a heavy chain constant region having the amino acid sequence of SEQ ID NO:105, and a light chain constant region having the amino acid sequence of SEQ ID NO:101.

36. The method of any one of claims 24-27, wherein the MCAM antagonist is a monoclonal antibody comprising a mature heavy chain variable region having the amino acid sequence of SEQ ID NO:93, a mature light chain variable region having the amino acid sequence of SEQ ID NO:94, a heavy chain constant region having the amino acid sequence of SEQ ID NO:104, and a light chain constant region having the amino acid sequence of SEQ ID NO:102.

37. The method of any one of claims 24-27, wherein the MCAM antagonist is a monoclonal antibody comprising a mature heavy chain variable region having the amino acid sequence of SEQ ID NO:93, a mature light chain variable region having the amino acid sequence of SEQ ID NO:94, a heavy chain constant region having the amino acid sequence of SEQ ID NO:105, and a light chain constant region having the amino acid sequence of SEQ ID NO:102.
